SEQUENCE LISTING (13 sequences)

SEQ ID NO: 1 - DNA, 1248 bp, synthetic construct
  source 1..1248: mol_type = other DNA
atggcggcgtccatttcgggctacaccttcagtgctgtgtgtttccacagcgccaacagcaacgcggaccacgaaggatttttactgggagaggtaagacaagaggaaacgtttagcatcagtgactcacaaatcagcaacacagaatttctgcaagtaattgaaatccataaccatcagccttgttcaaaactttttagtttttatgactacgcaagcaaagtgaatgaggagagtttggacaggattcttaaagatcggagaaagaaagtcattgggtggtacagattccggcgcaatacgcagcagcagatgtcctacagagagcaggttcttcacaagcagctcacccgcatcctcggcgtgcccgacctcgtctttcttctcttcagcttcatctccactgccaacaattccactcacgctttagaatatgtgctcttcagaccaaatagaaggtataatcagaggatatcactcgctattcccaatctaggaaatactagccagcaagagtacaaagtgtcttcagtgccaaatacttctcagagttatgccaaagtgattaaagaacatggtactgacttttttgacaaggatggagtgatgaaagacatcagggcgatttatcaggtttataatgcacttcaggagaaagttcaggcagtgtgtgcagatgttgaaaagagtgagcgagttgttgaatcttgtcaggcagaagtgaacaaattaagaagacaaatcactcagaggaaaaatgaaaaggaacaagaaagaagattgcagcaggcagtgttaagcagacagatgccgtctgaaagcttggacccagcgttcagtcctcggatgccgtcctctgggtttgcagctgaaggcagaagtacacttggagatgcagaggcctcggatcctcctcccccttactctgattttcacccaaacaatcaagaaagtactttgagccactctcgcatggaaaggagtgtctttatgcctcgacctcaagctgtgggctcttccaattatgcttccaccagtgccggactgaagtatcctggaagtggggctgaccttcctcctccccaaagagcagctggagacagtggtgaggattcagacgacagtgattatgaaaatttgattgaccctacagagccttctaatagtgaatactcacattcaaaggattctcgacccatggcacatcccgacgaggaccccaggaacactcagacctcccagatttaa

SEQ ID NO: 2 - protein, 416 aa, synthetic construct
TMAASISGYTFSAVCFHSANSNADHEGFLLGEVRQEETFSISDSQISNTEFLQVIEIHNHQPCSKLFSFYDYASKVNEESLDRILKDRRKKVIGWYRFRRNTQQQMSYREQVLHKQLTRILGVPDLVFLLFSFISTANNSTHALEYVLFRPNRRYNQRISLAIPNLGNTSQQEYKVSSVPNTSQSYAKVIKEHGTDFFDKDGVMKDIRAIYQVYNALQEKVQAVCADVEKSERVVESCQAEVNKLRRQITQRKNEKEQERRLQQAVLSRQMPSESLDPAFSPRMPSSGFAAEGRSTLGDAEASDPPPPYSDFHPNNQESTLSHSRMERSVFMPRPQAVGSSNYASTSAGLKYPGSGADLPPPQRAAGDSGEDSDDSDYENLIDPTEPSNSEYSHSKDSRPMAHPDEDPRNTQTSQI

SEQ ID NO: 3 - DNA, 951 bp, synthetic construct
  source 1..951: mol_type = other DNA
atggcggtgcaggtggtgcaggcggtgcaggcggttcatctcgagtctgacgctttcctcgtttgtctcaaccacgctctgagcacagagaaggaggaagtaatggggctgtgcataggggagttgaacgatgatacaaggagtgactccaaatttgcatatactggaactgaaatgcgcacagttgctgaaaaggttgatgccgtcagaattgttcacattcattctgtcatcatcttacgacgttctgataagaggaaggaccgagtagaaatttctccagagcagctgtctgcagcttcaacagaggcagagaggttggctgaactgacaggccgccccatgagagttgtgggctggtatcattcccatcctcatataactgtttggccttcacatgttgatgttcgcacacaagccatgtaccagatgatggatcaaggctttgtaggacttattttttcctgtttcatagaagataagaacacaaagactggccgggtactctacacttgcttccaatccatacaggcccaaaagagttcagagtcccttcatggtccacgagacttctggagctccagccagcacatctccattgagggccagaaggaagaggaaaggtatgagagaatcgaaatcccaatccatattgtacctcatgtcactatcgggaaagtgtgccttgaatcagcagtagagctgcccaagatcctgtgccaggaggagcaggatgcgtataggaggatccacagccttacacatctggactcagtaaccaagatccataatggctcagtgtttaccaagaatctgtgcagtcagatgtcggcagtcagcgggcctctcctacagtggttggaggacagactggagcaaaaccaacagcatttgcaggaattacaacaagaaaaggaagagcttatgcaagaactttcttctctagaataa

SEQ ID NO: 4 - protein, 316 aa, synthetic construct
MAVQVVQAVQAVHLESDAFLVCLNHALSTEKEEVMGLCIGELNDDTRSDSKFAYTGTEMRTVAEKVDAVRIVHIHSVIILRRSDKRKDRVEISPEQLSAASTEAERLAELTGRPMRVVGWYHSHPHITVWPSHVDVRTQAMYQMMDQGFVGLIFSCFIEDKNTKTGRVLYTCFQSIQAQKSSESLHGPRDFWSSSQHISIEGQKEEERYERIEIPIHIVPHVTIGKVCLESAVELPKILCQEEQDAYRRIHSLTHLDSVTKIHNGSVFTKNLCSQMSAVSGPLLQWLEDRLEQNQQHLQELQQEKEELMQELSSLE

SEQ ID NO: 5 - DNA, 1155 bp, synthetic construct
  source 1..1155: mol_type = other DNA
atgtccccagaagtggccttgaaccgaatatctccaatgctctcccctttcatatctagcgtggtccggaatggaaaagtgggactggatgctacaaactgtttgaggataactgacttaaaatctggctgcacatcattgactcctgggcccaactgtgaccgatttaaactgcacataccatatgctggagagacattaaagtgggatatcattttcaatgcccaatacccagaactgcctcccgattttatctttggagaagatgctgaattcctgccagacccctcagctttgcagaatcttgcctcctggaatccttcaaatcctgaatgtctcttacttgtggtgaaggaacttgtgcaacaatatcaccaattccaatgtagccgcctccgggagagctcccgcctcatgtttgaataccagacattactggaggagccacagtatggagagaacatggaaatttatgctgggaaaaaaaacaactggactggtgaattttcagctcgtttccttttgaagctgcccgtagatttcagcaatatccccacataccttctcaaggatgtaaatgaagaccctggagaagatgtggccctcctctctgttagttttgaggacactgaagccacccaggtgtaccccaagctgtacttgtcacctcgaattgagcatgcacttggaggctcctcagctcttcatatcccagcttttccaggaggaggatgtctcattgattacgttcctcaagtatgccacctgctcaccaacaaggtgcagtacgtgattcaagggtatcacaaaagaagagagtatattgctgcttttctcagtcactttggcacaggtgtcgtggaatatgatgcagaaggctttacaaaactcactctgctgctgatgtggaaagatttttgttttcttgtacacattgacctgcctctgtttttccctcgagaccagccaactctcacatttcagtccgtttatcactttaccaacagtggacagctttactcccaggcccaaaaaaattatccatacagccccagatgggatggaaatgaaatggccaaaagagcaaaggcttatttcaaaacctttgtccctcagttccaggaggcagcatttgccaatggaaagctctagtaa

SEQ ID NO: 6 - protein, 383 aa, synthetic construct
MSPEVALNRISPMLSPFISSVVRNGKVGLDATNCLRITDLKSGCTSLTPGPNCDRFKLHIPYAGETLKWDIIFNAQYPELPPDFIFGEDAEFLPDPSALQNLASWNPSNPECLLLVVKELVQQYHQFQCSRLRESSRLMFEYQTLLEEPQYGENMEIYAGKKNNWTGEFSARFLLKLPVDFSNIPTYLLKDVNEDPGEDVALLSVSFEDTEATQVYPKLYLSPRIEHALGGSSALHIPAFPGGGCLIDYVPQVCHLLTNKVQYVIQGYHKRREYIAAFLSHFGTGVVEYDAEGFTKLTLLLMWKDFCFLVHIDLPLFFPRDQPTLTFQSVYHFTNSGQLYSQAQKNYPYSPRWDGNEMAKRAKAYFKTFVPQFQEAAFANGKL

SEQ ID NO: 7 - DNA, 990 bp, synthetic construct
  source 1..990: mol_type = other DNA
atggaagtggcagagcccagcagccccactgaagaggaggaggaggaagaggagcactcggcagagcctcggccccgcactcgctccaatcctgaaggggctgaggaccgggcagtaggggcacaggccagcgtgggcagccgcagcgagggtgagggtgaggccgccagtgctgatgatgggagcctcaacacttcaggagccggccctaagtcctggcaggtgcccccgccagcccctgaggtccaaattcggacaccaagggtcaactgtccagagaaagtgattatctgcctggacctgtcagaggaaatgtcactgccaaagctggagtcgttcaacggctccaaaaccaacgccctcaatgtctcccagaagatgattgagatgttcgtgcggacaaaacacaagatcgacaaaagccacgagtttgcactggtggtggtgaacgatgacacggcctggctgtctggcctgacctccgacccccgcgagctctgtagctgcctctatgatctggagacggcctcctgttccaccttcaatctggaaggacttttcagcctcatccagcagaaaactgagcttccggtcacagagaacgtgcagacgattcccccgccatatgtggtccgcaccatccttgtctacagccgtccaccttgccagccccagttctccttgacggagcccatgaagaaaatgttccagtgcccatatttcttctttgacgttgtttacatccacaatggcactgaggagaaggaggaggagatgagttggaaggatatgtttgccttcatgggcagcctggataccaagggtaccagctacaagtatgaggtggcactggctgggccagccctggagttgcacaactgcatggcgaaactgttggcccaccccctgcagcggccttgccagagccatgcttcctacagcctgctggaggaggaggatgaagccattgaggttgaggccactgtctga

SEQ ID NO: 8 - protein, 329 aa, synthetic construct
MEVAEPSSPTEEEEEEEEHSAEPRPRTRSNPEGAEDRAVGAQASVGSRSEGEGEAASADDGSLNTSGAGPKSWQVPPPAPEVQIRTPRVNCPEKVIICLDLSEEMSLPKLESFNGSKTNALNVSQKMIEMFVRTKHKIDKSHEFALVVVNDDTAWLSGLTSDPRELCSCLYDLETASCSTFNLEGLFSLIQQKTELPVTENVQTIPPPYVVRTILVYSRPPCQPQFSLTEPMKKMFQCPYFFFDVVYIHNGTEEKEEEMSWKDMFAFMGSLDTKGTSYKYEVALAGPALELHNCMAKLLAHPLQRPCQSHASYSLLEEEDEAIEVEATV

SEQ ID NO: 9 - DNA, 5108 bp, synthetic construct
  source 1..5108: mol_type = other DNA
taatgtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttactagtcactagtttcggttaccgtgaaggttttggtagcgtcgtcgtaggtccattcaccgtcaacaccgttgtcgttagcgtactgtttgaaaactttttccgcggtagcagcgtcaacagcttcggtggtggtttcacctttcagggttttaccgttcagagcaagcttgtactgagccatggcgcccgacgacttgtacagctcgtccatgccaccaccaaggttgctgtatttggcaactgccacttctctttgcactacgtaggtttcattgtcactctcctcgatcctttccaggcggtgatcaacggcatggacaccgggcatcttgatgttcccgacggtcttttttgacctgtaagttgtcacgaagttgcagtgcagatggccaccaccatcaactttcagtgcgatgtcagtgtatcctctcagaccaccatctgctggatacatcatctctgtattaggctcccaacccttggtcttcttctgcatcacgggaccattggagggaaagtttacccctctgaccttgacgttgtagacgagctcgccatcctcaaggctggtgtcctgcgtgacggtgacgactccaccatcttcgtatctcgtaactctttcccaagtaaaaccctcaggaaaggactgtttaaagaaatcagggatgtcggccgggtacttgataaaggtacggctgccatacatgaacgacgtggcaagaatgtcaaaggcaaatggcaggggtcctccctcgatgactttgatcctcatggtttgcactccctcgtacggtctgccttctccttcacctgtgcatttgaattggtggccgttgaccgaaccttccatgaccaccttcatacgcatattttccttgatcagctcttcgcccttagacaccatgcccgacgatcctgtttcgggtaatggcccctggaacagaacttccaggccgagctttccattggcaaatgctgcctcctggaactgagggacaaaggttttgaaataagcctttgctcttttggccatttcatttccatcccatctggggctgtatggataatttttttgggcctgggagtaaagctgtccactgttggtaaagtgataaacggactgaaatgtgagagttggctggtctcgagggaaaaacagaggcaggtcaatgtgtacaagaaaacaaaaatctttccacatcagcagcagagtgagttttgtaaagccttctgcatcatattccacgacacctgtgccaaagtgactgagaaaagcagcaatatactctcttcttttgtgatacccttgaatcacgtactgcaccttgttggtgagcaggtggcatacttgaggaacgtaatcaatgagacatcctcctcctggaaaagctgggatatgaagagctgaggagcctccaagtgcatgctcaattcgaggtgacaagtacagcttggggtacacctgggtggcttcagtgtcctcaaaactaacagagaggagggccacatcttctccagggtcttcatttacatccttgagaaggtatgtggggatattgctgaaatctacgggcagcttcaaaaggaaacgagctgaaaattcaccagtccagttgttttttttcccagcataaatttccatgttctctccatactgtggctcctccagtaatgtctggtattcaaacatgaggcgggagctctcccggaggcggctacattggaattggtgatattgttgcacaagttccttcaccacaagtaagagacattcaggatttgaaggattccaggaggcaagattctgcaaagctgaggggtctggcaggaattcagcatcttctccaaagataaaatcgggaggcagttctgggtattgggcattgaaaatgatatcccactttaatgtctctccagcatatggtatgtgcagtttaaatcggtcacagttgggcccaggagtcaatgatgtgcagccagattttaagtcagttatcctcaaacagtttgtagcatccagtcccacttttccattccggaccacgctagatatgaaaggggagagcattggagatattcggttcaaggccacttctggggacatggcgccctgaaaatacaggttttcggtcgttgggatatcgtaatcgtgatggtgatggtgatggtagtacgagctaccctcgtgccactcgatcttctgggcctcgaagatatcgttcaggccgcccatggtctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacaccatggcggtgcaggtggtgcaggcggtgcaggcggttcatctcgagtctgacgctttcctcgtttgtctcaaccacgctctgagcacagagaaggaggaagtaatggggctgtgcataggggagttgaacgatgatacaaggagtgactccaaatttgcatatactggaactgaaatgcgcacagttgctgaaaaggttgatgccgtcagaattgttcacattcattctgtcatcatcttacgacgttctgataagaggaaggaccgagtagaaatttctccagagcagctgtctgcagcttcaacagaggcagagaggttggctgaactgacaggccgccccatgagagttgtgggctggtatcattcccatcctcatataactgtttggccttcacatgttgatgttcgcacacaagccatgtaccagatgatggatcaaggctttgtaggacttattttttcctgtttcatagaagataagaacacaaagactggccgggtactctacacttgcttccaatccatacaggcccaaaagagttcagagtcccttcatggtccacgagacttctggagctccagccagcacatctccattgagggccagaaggaagaggaaaggtatgagagaatcgaaatcccaatccatattgtacctcatgtcactatcgggaaagtgtgccttgaatcagcagtagagctgcccaagatcctgtgccaggaggagcaggatgcgtataggaggatccacagccttacacatctggactcagtaaccaagatccataatggctcagtgtttaccaagaatctgtgcagtcagatgtcggcagtcagcgggcctctcctacagtggttggaggacagactggagcaaaaccaacagcatttgcaggaattacaacaagaaaaggaagagcttatgcaagaactttcttctctagaaggcctggaagttctgttccaggggccaaaccacgtttcgtcgggcatggtgagcaagggcgaggagctgttcaccggggtggtgcccatcctggtcgagctggacggcgacgtaaacggccacaagttcagcgtccgcggcgagggcgagggcgatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtcgtcggactacaaagaccatgacggtgattataaagatcatgacatcgattacaaggatgacgatgacaagtagtaactgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggtaat

SEQ ID NO: 10 - DNA, 4025 bp, synthetic construct
  source 1..4025: mol_type = other DNA
taatgtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttactacttgtcatcgtcatccttgtaatcgatgtcatgatctttataatcaccgtcatggtctttgtagtccgacgacttgtacagctcgtccatgccatgtgtaatcccggcggcggtcacgaactccagcaggaccatgtgatcgcgcttctcgttggggtctttgctcagcttggactgatggctcaggtagtggttgtcgggcagcagcacggggccgtcgccgatgggggtgttctgctggtagtggtcggcgagctgcacgctgccgtcctcaacgttgtggcggatcttgaagttagccttgatgcagttcttctgcttgtcggccgtgatatagacgtagtggctgttgaagttgtactccagcttgtgccccaggatgttgccgtcctccttgaagtcgatgcccttcagctcgatgcggttcaccagggtgtcgccctcgaacttcacctcggcgcgggtcttgtaggtaccgtcgtccttgaaagagatggtgcgctcctggacgtagccttcgggcatggcggacttgaagaagtcgtgctgcttcatgtggtcggggtagcggctgaagcaggccacgccgtagccgaaggtggtcacgagggtgggccagggcacgggcagcttgccggtggtgcagatgaacttcagggtcagcttgccgttggtggcatcgccctcgccctcgccgcggacgctgaacttgtggccgtttacgtcgccgtccagctcgaccaggatgggcaccaccccggtgaacagctcctcgcccttgctcaccatgcccgacgaaacgtggtttggcccctggaacagaacttccaggccgagctttccattggcaaatgctgcctcctggaactgagggacaaaggttttgaaataagcctttgctcttttggccatttcatttccatcccatctggggctgtatggataatttttttgggcctgggagtaaagctgtccactgttggtaaagtgataaacggactgaaatgtgagagttggctggtctcgagggaaaaacagaggcaggtcaatgtgtacaagaaaacaaaaatctttccacatcagcagcagagtgagttttgtaaagccttctgcatcatattccacgacacctgtgccaaagtgactgagaaaagcagcaatatactctcttcttttgtgatacccttgaatcacgtactgcaccttgttggtgagcaggtggcatacttgaggaacgtaatcaatgagacatcctcctcctggaaaagctgggatatgaagagctgaggagcctccaagtgcatgctcaattcgaggtgacaagtacagcttggggtacacctgggtggcttcagtgtcctcaaaactaacagagaggagggccacatcttctccagggtcttcatttacatccttgagaaggtatgtggggatattgctgaaatctacgggcagcttcaaaaggaaacgagctgaaaattcaccagtccagttgttttttttcccagcataaatttccatgttctctccatactgtggctcctccagtaatgtctggtattcaaacatgaggcgggagctctcccggaggcggctacattggaattggtgatattgttgcacaagttccttcaccacaagtaagagacattcaggatttgaaggattccaggaggcaagattctgcaaagctgaggggtctggcaggaattcagcatcttctccaaagataaaatcgggaggcagttctgggtattgggcattgaaaatgatatcccactttaatgtctctccagcatatggtatgtgcagtttaaatcggtcacagttgggcccaggagtcaatgatgtgcagccagattttaagtcagttatcctcaaacagtttgtagcatccagtcccacttttccattccggaccacgctagatatgaaaggggagagcattggagatattcggttcaaggccacttctggggacatggtctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacaccatggcggtgcaggtggtgcaggcggtgcaggcggttcatctcgagtctgacgctttcctcgtttgtctcaaccacgctctgagcacagagaaggaggaagtaatggggctgtgcataggggagttgaacgatgatacaaggagtgactccaaatttgcatatactggaactgaaatgcgcacagttgctgaaaaggttgatgccgtcagaattgttcacattcattctgtcatcatcttacgacgttctgataagaggaaggaccgagtagaaatttctccagagcagctgtctgcagcttcaacagaggcagagaggttggctgaactgacaggccgccccatgagagttgtgggctggtatcattcccatcctcatataactgtttggccttcacatgttgatgttcgcacacaagccatgtaccagatgatggatcaaggctttgtaggacttattttttcctgtttcatagaagataagaacacaaagactggccgggtactctacacttgcttccaatccatacaggcccaaaagagttcagagtcccttcatggtccacgagacttctggagctccagccagcacatctccattgagggccagaaggaagaggaaaggtatgagagaatcgaaatcccaatccatattgtacctcatgtcactatcgggaaagtgtgccttgaatcagcagtagagctgcccaagatcctgtgccaggaggagcaggatgcgtataggaggatccacagccttacacatctggactcagtaaccaagatccataatggctcagtgtttaccaagaatctgtgcagtcagatgtcggcagtcagcgggcctctcctacagtggttggaggacagactggagcaaaaccaacagcatttgcaggaattacaacaagaaaaggaagagcttatgcaagaactttcttctctagaatagtaactgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggtaat

SEQ ID NO: 11 - DNA, 4274 bp, synthetic construct
  source 1..4274: mol_type = other DNA
taatgtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttactagtcactagtttcggttaccgtgaaggttttggtagcgtcgtcgtaggtccattcaccgtcaacaccgttgtcgttagcgtactgtttgaaaactttttccgcggtagcagcgtcaacagcttcggtggtggtttcacctttcagggttttaccgttcagagcaagcttgtactgagccatggcgcccgacgacttgtacagctcgtccatgccaccaccaaggttgctgtatttggcaactgccacttctctttgcactacgtaggtttcattgtcactctcctcgatcctttccaggcggtgatcaacggcatggacaccgggcatcttgatgttcccgacggtcttttttgacctgtaagttgtcacgaagttgcagtgcagatggccaccaccatcaactttcagtgcgatgtcagtgtatcctctcagaccaccatctgctggatacatcatctctgtattaggctcccaacccttggtcttcttctgcatcacgggaccattggagggaaagtttacccctctgaccttgacgttgtagacgagctcgccatcctcaaggctggtgtcctgcgtgacggtgacgactccaccatcttcgtatctcgtaactctttcccaagtaaaaccctcaggaaaggactgtttaaagaaatcagggatgtcggccgggtacttgataaaggtacggctgccatacatgaacgacgtggcaagaatgtcaaaggcaaatggcaggggtcctccctcgatgactttgatcctcatggtttgcactccctcgtacggtctgccttctccttcacctgtgcatttgaattggtggccgttgaccgaaccttccatgaccaccttcatacgcatattttccttgatcagctcttcgcccttagacaccatgcccgacgatcctgtttcgggtaatggcccctggaacagaacttccaggccgagctttccattggcaaatgctgcctcctggaactgagggacaaaggttttgaaataagcctttgctcttttggccatttcatttccatcccatctggggctgtatggataatttttttgggcctgggagtaaagctgtccactgttggtaaagtgataaacggactgaaatgtgagagttggctggtctcgagggaaaaacagaggcaggtcaatgtgtacaagaaaacaaaaatctttccacatcagcagcagagtgagttttgtaaagccttctgcatcatattccacgacacctgtgccaaagtgactgagaaaagcagcaatatactctcttcttttgtgatacccttgaatcacgtactgcaccttgttggtgagcaggtggcatacttgaggaacgtaatcaatgagacatcctcctcctggaaaagctgggatatgaagagctgaggagcctccaagtgcatgctcaattcgaggtgacaagtacagcttggggtacacctgggtggcttcagtgtcctcaaaactaacagagaggagggccacatcttctccagggtcttcatttacatccttgagaaggtatgtggggatattgctgaaatctacgggcagcttcaaaaggaaacgagctgaaaattcaccagtccagttgttttttttcccagcataaatttccatgttctctccatactgtggctcctccagtaatgtctggtattcaaacatgaggcgggagctctcccggaggcggctacattggaattggtgatattgttgcacaagttccttcaccacaagtaagagacattcaggatttgaaggattccaggaggcaagattctgcaaagctgaggggtctggcaggaattcagcatcttctccaaagataaaatcgggaggcagttctgggtattgggcattgaaaatgatatcccactttaatgtctctccagcatatggtatgtgcagtttaaatcggtcacagttgggcccaggagtcaatgatgtgcagccagattttaagtcagttatcctcaaacagtttgtagcatccagtcccacttttccattccggaccacgctagatatgaaaggggagagcattggagatattcggttcaaggccacttctggggacatggcgccctgaaaatacaggttttcggtcgttgggatatcgtaatcgtgatggtgatggtgatggtagtacgagctaccctcgtgccactcgatcttctgggcctcgaagatatcgttcaggccgcccatggtctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacaccatggcggtgcaggtggtgcaggcggtgcaggcggttcatctcgagtctgacgctttcctcgtttgtctcaaccacgctctgagcacagagaaggaggaagtaatggggctgtgcataggggagttgaacgatgatacaaggagtgactccaaatttgcatatactggaactgaaatgcgcacagttgctgaaaaggttgatgccgtcagaattgttcacattcattctgtcatcatcttacgacgttctgataagaggaaggaccgagtagaaatttctccagagcagctgtctgcagcttcaacagaggcagagaggttggctgaactgacaggccgccccatgagagttgtgggctggtatcattcccatcctcatataactgtttggccttcacatgttgatgttcgcacacaagccatgtaccagatgatggatcaaggctttgtaggacttattttttcctgtttcatagaagataagaacacaaagactggccgggtactctacacttgcttccaatccatacaggcccaaaagagttcagagtcccttcatggtccacgagacttctggagctccagccagcacatctccattgagggccagaaggaagaggaaaggtatgagagaatcgaaatcccaatccatattgtacctcatgtcactatcgggaaagtgtgccttgaatcagcagtagagctgcccaagatcctgtgccaggaggagcaggatgcgtataggaggatccacagccttacacatctggactcagtaaccaagatccataatggctcagtgtttaccaagaatctgtgcagtcagatgtcggcagtcagcgggcctctcctacagtggttggaggacagactggagcaaaaccaacagcatttgcaggaattacaacaagaaaaggaagagcttatgcaagaactttcttctctagaatagtaactgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggtaat

SEQ ID NO: 12 - DNA, 4061 bp, synthetic construct
  source 1..4061: mol_type = other DNA
taatgtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttactaggtgataccggcagcatttacatactcatgaaggaccatgtggtcacgcatgcccgacgagccgcccgacgagccgagctttccattggcaaatgctgcctcctggaactgagggacaaaggttttgaaataagcctttgctcttttggccatttcatttccatcccatctggggctgtatggataatttttttgggcctgggagtaaagctgtccactgttggtaaagtgataaacggactgaaatgtgagagttggctggtctcgagggaaaaacagaggcaggtcaatgtgtacaagaaaacaaaaatctttccacatcagcagcagagtgagttttgtaaagccttctgcatcatattccacgacacctgtgccaaagtgactgagaaaagcagcaatatactctcttcttttgtgatacccttgaatcacgtactgcaccttgttggtgagcaggtggcatacttgaggaacgtaatcaatgagacatcctcctcctggaaaagctgggatatgaagagctgaggagcctccaagtgcatgctcaattcgaggtgacaagtacagcttggggtacacctgggtggcttcagtgtcctcaaaactaacagagaggagggccacatcttctccagggtcttcatttacatccttgagaaggtatgtggggatattgctgaaatctacgggcagcttcaaaaggaaacgagctgaaaattcaccagtccagttgttttttttcccagcataaatttccatgttctctccatactgtggctcctccagtaatgtctggtattcaaacatgaggcgggagctctcccggaggcggctacattggaattggtgatattgttgcacaagttccttcaccacaagtaagagacattcaggatttgaaggattccaggaggcaagattctgcaaagctgaggggtctggcaggaattcagcatcttctccaaagataaaatcgggaggcagttctgggtattgggcattgaaaatgatatcccactttaatgtctctccagcatatggtatgtgcagtttaaatcggtcacagttgggcccaggagtcaatgatgtgcagccagattttaagtcagttatcctcaaacagtttgtagcatccagtcccacttttccattccggaccacgctagatatgaaaggggagagcattggagatattcggttcaaggccacttctggggacatggcgccctgaaaatacaggttttcggtcgttgggatatcgtaatcgtgatggtgatggtgatggtagtacgagctaccctcgtgccactcgatcttctgggcctcgaagatatcgttcaggccgcccatggtctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacaccatggcggtgcaggtggtgcaggcggtgcaggcggttcatctcgagtctgacgctttcctcgtttgtctcaaccacgctctgagcacagagaaggaggaagtaatggggctgtgcataggggagttgaacgatgatacaaggagtgactccaaatttgcatatactggaactgaaatgcgcacagttgctgaaaaggttgatgccgtcagaattgttcacattcattctgtcatcatcttacgacgttctgataagaggaaggaccgagtagaaatttctccagagcagctgtctgcagcttcaacagaggcagagaggttggctgaactgacaggccgccccatgagagttgtgggctggtatcattcccatcctcatataactgtttggccttcacatgttgatgttcgcacacaagccatgtaccagatgatggatcaaggctttgtaggacttattttttcctgtttcatagaagataagaacacaaagactggccgggtactctacacttgcttccaatccatacaggcccaaaagagttcagagtcccttcatggtccacgagacttctggagctccagccagcacatctccattgagggccagaaggaagaggaaaggtatgagagaatcgaaatcccaatccatattgtacctcatgtcactatcgggaaagtgtgccttgaatcagcagtagagctgcccaagatcctgtgccaggaggagcaggatgcgtataggaggatccacagccttacacatctggactcagtaaccaagatccataatggctcagtgtttaccaagaatctgtgcagtcagatgtcggcagtcagcgggcctctcctacagtggttggaggacagactggagcaaaaccaacagcatttgcaggaattacaacaagaaaaggaagagcttatgcaagaactttcttctctagaaggctcgtcgggcggctcgtcgggcatgtccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaatagtaactgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggtaat

SEQ ID NO: 13 - DNA, 3320 bp, synthetic construct
  source 1..3320: mol_type = other DNA
taatgtttaaacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcttactagagctttccattggcaaatgctgcctcctggaactgagggacaaaggttttgaaataagcctttgctcttttggccatttcatttccatcccatctggggctgtatggataatttttttgggcctgggagtaaagctgtccactgttggtaaagtgataaacggactgaaatgtgagagttggctggtctcgagggaaaaacagaggcaggtcaatgtgtacaagaaaacaaaaatctttccacatcagcagcagagtgagttttgtaaagccttctgcatcatattccacgacacctgtgccaaagtgactgagaaaagcagcaatatactctcttcttttgtgatacccttgaatcacgtactgcaccttgttggtgagcaggtggcatacttgaggaacgtaatcaatgagacatcctcctcctggaaaagctgggatatgaagagctgaggagcctccaagtgcatgctcaattcgaggtgacaagtacagcttggggtacacctgggtggcttcagtgtcctcaaaactaacagagaggagggccacatcttctccagggtcttcatttacatccttgagaaggtatgtggggatattgctgaaatctacgggcagcttcaaaaggaaacgagctgaaaattcaccagtccagttgttttttttcccagcataaatttccatgttctctccatactgtggctcctccagtaatgtctggtattcaaacatgaggcgggagctctcccggaggcggctacattggaattggtgatattgttgcacaagttccttcaccacaagtaagagacattcaggatttgaaggattccaggaggcaagattctgcaaagctgaggggtctggcaggaattcagcatcttctccaaagataaaatcgggaggcagttctgggtattgggcattgaaaatgatatcccactttaatgtctctccagcatatggtatgtgcagtttaaatcggtcacagttgggcccaggagtcaatgatgtgcagccagattttaagtcagttatcctcaaacagtttgtagcatccagtcccacttttccattccggaccacgctagatatgaaaggggagagcattggagatattcggttcaaggccacttctggggacatggcgccctgaaaatacaggttttcggtcgttgggatatcgtaatcgtgatggtgatggtgatggtagtacgagctaccctcgtgccactcgatcttctgggcctcgaagatatcgttcaggccgcccatggtctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactagtatcgattcgcgacctactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacaccatggcggtgcaggtggtgcaggcggtgcaggcggttcatctcgagtctgacgctttcctcgtttgtctcaaccacgctctgagcacagagaaggaggaagtaatggggctgtgcataggggagttgaacgatgatacaaggagtgactccaaatttgcatatactggaactgaaatgcgcacagttgctgaaaaggttgatgccgtcagaattgttcacattcattctgtcatcatcttacgacgttctgataagaggaaggaccgagtagaaatttctccagagcagctgtctgcagcttcaacagaggcagagaggttggctgaactgacaggccgccccatgagagttgtgggctggtatcattcccatcctcatataactgtttggccttcacatgttgatgttcgcacacaagccatgtaccagatgatggatcaaggctttgtaggacttattttttcctgtttcatagaagataagaacacaaagactggccgggtactctacacttgcttccaatccatacaggcccaaaagagttcagagtcccttcatggtccacgagacttctggagctccagccagcacatctccattgagggccagaaggaagaggaaaggtatgagagaatcgaaatcccaatccatattgtacctcatgtcactatcgggaaagtgtgccttgaatcagcagtagagctgcccaagatcctgtgccaggaggagcaggatgcgtataggaggatccacagccttacacatctggactcagtaaccaagatccataatggctcagtgtttaccaagaatctgtgcagtcagatgtcggcagtcagcgggcctctcctacagtggttggaggacagactggagcaaaaccaacagcatttgcaggaattacaacaagaaaaggaagagcttatgcaagaactttcttctctagaatagtaactgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctagaagatccggctgctaacaaagcccgaaaggaagctgagttggctgctgccaccgctgagcaataactatcataacccctaggtaat

(19) United States
(12) Patent Application Publication
(10) Pub. No.: US 2023/0317203 A1
(43) Pub. Date: Oct. 5, 2023

(54) SCREENING METHOD

(71) Applicant: University of Leeds, Leeds (GB)

(72) Inventors: Elton Zeqiraj, Leeds (GB); Miriam Walden, Norwich (GB); Francesca Chandler, Leeds (GB)

(73) Assignee: University of Leeds, Leeds (GB)

(21) Appl. No.: 18/194,615

(22) Filed: Mar. 31, 2023

Related U.S. Application Data
(60) Provisional application No. 63/326,502, filed on Apr. 1, 2022.

(51) Int. Cl. G16B 15/30; G16B 30/00; G01N 23/20008

(52) U.S. Cl. CPC G16B 15/30; G16B 30/00; G01N 23/20008; G01N 2223/304; G01N 2223/612

(57) ABSTRACT

The present invention relates to method of identifying a selective BRISC inhibitor. The present invention also relates to a stable BRISC dimer. The present invention further relates to use of the stable BRISC dimer to generate cryo-Electron Microscopy (cryo-EM), crystallography, nuclear magnetic resonance and/or X-ray crystallography structures for structure guided drug design.

### RELATED APPLICATIONS

This application claims priority to U.S. provisional application Ser. No. 63/326,502, filed Apr. 1, 2022, the entire content of which is incorporated herein by reference.

### REFERENCE TO AN ELECTRONIC SEQUENCE LISTING

The contents of the electronic sequence listing (H066470100US01-SEQ-GIC.xml; Size: 38,791 bytes; and Date of Creation: Mar. 31, 2023) is herein incorporated by reference in its entirety.

### FIELD

The invention relates to a method of screening for specific BRISC (BRCC36 isopeptidase complex) inhibitors. The invention also relates to stable BRISC dimers that may be used for sample preparation in electron microscopy (EM), crystallography or Nuclear Magnetic Resonance (NMR) studies.

### BACKGROUND

Ubiquitin-dependent signalling regulates almost all cellular signalling pathways. The removal of ubiquitin is catalysed by deubiquitylating enzymes, commonly referred to as DUBs. There are over 100 human DUBs, which control ubiquitin-mediated signal transduction by dictating the activity, localisation or stability of a protein substrate¹. DUB dysfunction is in general implicated in a range of pathologies, including autoimmune disorders, oncology and neurodegeneration²⁻⁴. Consequently, DUBs are an attractive therapeutic target and the focus of many drug discovery efforts in the last decade^{5,6}.

Exploiting components of the ubiquitin-proteasome system (UPS) for therapeutic benefit is therefore of major pharmacological interest. The BRCC36 isopeptidase complex (BRISC) DUB in particular regulates inflammatory cytokine signalling by selectively cleaving K63-linked polyubiquitin chains on Type I interferon receptors, with implications in autoimmune disease.

It is known that the cytoplasmic BRISC DUB complex removes lysine-63-linked ubiquitin chains from interferon receptors IFNAR1 and 2 and is required for maximal interferon responses. BRISC interacts with the metabolic enzyme serine hydroxymethyl transferase (SHMT2) which in turns regulates IFNAR1/2 endosomal-lysosomal degradation and inflammation. The BRISC complex contains a deubiquitylase (BRCC36) and accessory subunits (Abraxas2, BRCC45, MERIT40), which form a U-shaped structure and have a 2:2:2:2 stoichiometry. BRCC36 is the active subunit and belongs to the Zn²⁺-dependent JAMM domain DUBs.

The BRCC36 DUB is a member of the JAMM (JAB1, MOV34, and MPR1, Pad1 N-terminal (MPN)) metalloenzyme family and specifically cleaves lysine-63-linked (K63-Ub) chains^{7,8}. BRCC36 is present in two subcellular locations in two distinct macromolecular assemblies: a cytoplasmic BRCC36 isopeptidase complex (BRISC), and the nuclear Abraxas1 isopeptidase complex (ARISC). The BRISC complex regulates type I interferon signalling by deubiquitylating and stabilising IFNAR1/2 receptors, whilst the ARISC complex is targeted to double-stranded DNA breaks to facilitate breast cancer suppression⁹⁻¹¹. BRCC36 (MPN⁺) forms a stable heterodimeric complex with a pseudo-DUB partner, either with Abraxas1 (MPN⁻) in the nucleus, or Abraxas2 (MPN⁻) in the cytoplasm. The formation of an MPN⁺/MPN⁻ heterodimer is required for BRCC36 DUB activity¹². The BRISC and ARISC complexes are also composed of two additional accessory proteins, BRCC45 and MERIT40, to form dimer of tetramer complexes with a 2:2:2:2 stoichiometry. These eight-subunit enzyme complexes require further binding partners for cellular function. BRISC forms a complex with a metabolic enzyme, serine hydroxymethyltransferase (SHMT2) for targeting to IFNAR1/2 receptors and loss of this interaction leads to a reduction in interferon signalling¹³. Moreover, ARISC forms the BRCA1-A complex with BRCA1 and RAP80 in the nucleus to facilitate DNA damage repair⁹⁻¹¹. Recent cryo-electron microscopy structures of the BRISC-SHMT2 complex revealed that BRISC forms a distinct U-shaped assembly, with the BRCC36-Abraxas2 heterodimer bridging two BRCC45-MERIT40 “arms”, similar to ARISC structures¹³⁻¹⁵.

BRISC-mediated deubiquitylation of IFNAR1/2 receptors promotes JAK/STAT signalling and expression of interferon(IFN)-induced genes¹⁶. Elevated levels of type I IFN have long been observed in patients with autoimmune diseases, including systemic lupus erythematosus (SLE)¹⁷, rheumatoid arthritis (RA)¹⁸, and systemic scleroderma (SSc)¹⁹. BRISC-deficient mice are protected from elevated interferon signalling and chronic inflammation¹⁶. The above thus emphasises the importance of BRISC as a therapeutic target. Being able to target BRISC with small molecule inhibitors would provide a much needed therapeutic strategy to alleviate hyperactive cytokine signalling and autoimmune disease pathology.

Problematically, there are currently no small molecule inhibitors of the BRISC DUB complex. Most small-molecule inhibitors of the JAMM/MPN family of DUBs are broad-spectrum zinc chelators, such as 1,10-phenanthroline and thiolutin^{7,20}. More selective inhibitors have been described, including capzimin, a quinoline-8-thiol (8TQ) derivative which targets the active site zinc of proteasomal subunit Rpn11, but this still inhibits BRCC36 and AMSH at micromolar concentrations²¹. Inhibitors of the JAMM domain containing-COP9 signalosome (CSN) subunit, CSN5, also engage the catalytic zinc, but show specificity for CSN5 over AMSH and Rpn11²². Thus, whilst major progress has been made in DUB inhibitor development²³, small-molecule inhibitors of the JAMM/MPN DUBs exclusively target the conserved zinc binding pocket, which makes the development of selective inhibitors challenging.

Discovering selective inhibitors for Zn-dependent metalloenzymes and enzyme complexes such as BRISC is inherently difficult due to cross reactivity of small molecule inhibitors with other metalloenzymes. In addition, the ability to obtain high resolution structures by X-ray crystallography and/or electron microscopy to aid inhibitor design is difficult and no crystallisation conditions have been reported for the full BRISC complex or human BRCC36 which represents the active subunit. Similarly, cryoEM structures of BRISC alone have not been successful so far because of conformational flexibility and complex dissociation.

An alternative strategy to achieve selective DUB inhibition has been to exploit the substrate binding pocket with ubiquitin variant (UbV) inhibitors²⁴. This biologics approach has been applied to the STAMBP, JAMM/MPN DUBs, yielding specificity over SIAMBPL1²⁵.

### BRIEF SUMMARY OF THE DISCLOSURE

The present invention aims to solve the above issues. Firstly, the present invention solves the issue of discovering selective inhibitors of the BRISC DUB complex. Secondly, the present invention also provides stable BRISC dimers to address the issue of obtaining structures to aid inhibitor design.

The screening methods disclosed herein allow for the identification of first-in-class BRISC DUB inhibitors based on the use of an integrative structural biology approach which uncovered a unique mode of inhibition. In particular, the present invention was based on studies using complementary structural biology techniques which revealed an unexpected mode of target engagement and molecular basis for inhibition. Such study yielded cryo-electron microscopy (cryo-EM) structures for BRISC-inhibitor co-structures at 3.5 Å and 4.1 Å resolution revealing a new BRISC conformer, induced by inhibitor binding. The new conformation reveals a unique mode of action which led to the screening method and the stable BRISC dimers disclosed herein. The cryo-EM structures also revealed a unique binding site which make the inhibitors selective for BRISC unlike other inhibitors previously described in the prior art. The inhibitors identified through the screening methods act as “BRISC molecular glues (BLUEs)” between three BRISC subunits, impelling two BRISC monomers to form a dimeric complex, inhibiting BRISC DUB activity by sterically blocking the active site and interactions with the recruiting subunit SHMT2.

Advantageously, the screening methods disclosed herein allow for the identification of compounds that selectively inhibit the BRISC DUB complex in contrast to screening methods of the prior art. This is achievable through the detection of the dimeric complex which forms upon allosteric binding of a specific BRISC inhibitor. Techniques for detection of the dimeric complex include native mass spectrometry or mass photometry which advantageously enables high throughput screening. Furthermore, detection of dimeric BRISC complexes can be achieved by measuring the signal generated by Fluorescent resonance energy transfer (FRET) and bioluminescent resonance energy transfer (BRET) pairs labelled at specific subunits of BRISC. This method enables an even higher throughout and cheaper method to identify selective BRISC inhibitors.

Advantageously, the BRISC dimers disclosed lead to a more stable protein sample. Such stable sample is more amenable to crystallisation and/or cryoEM sample preparation allowing the determination of co-structures with BRISC and selective BRISC inhibitors (also referred herein as BRISC molecule glues or BLUES) which aids inhibitor design.

In accordance with the present invention there is provided a method of identifying a selective BRISC inhibitor comprising:
(i) contacting a population of BRISC monomers with a test compound; and
(ii) measuring the level of BRISC dimers before and after step (i),
wherein an increase in the level of BRISC dimers after contacting the population of BRISC monomers with the test compound identifies the test compound as a selective BRISC inhibitor.

In the method of the invention, the BRISC monomers may comprise an octameric complex comprising the subunits, Abraxas2, BRCC36, BRCC45 and MERIT40 at a 2:2:2:2 ratio.

In the method of the invention, the BRISC dimers may comprise a 16-meric complex comprising the subunits, Abraxas2, BRCC36, BRCC45 and MERIT40 at a 4:4:4:4 ratio.

In the method of the invention, the Abraxas2 subunit may be encoded by a nucleic acid sequence that is at least 85%, 90%, 95%, 96%, 97%, 98%, or 99% identical to the sequence of SEQ ID NO: 1.

In the method of the invention, the BRCC36 subunit may be encoded by a nucleic acid sequence that is at least 85%, 90%, 95%, 96%, 97%, 98%, or 99% identical to the sequence of SEQ ID NO: 3.

In the method of the invention, the BRCC45 subunit may be encoded by a nucleic acid sequence that is at least 85%, 90%, 95%, 96%, 97%, 98%, or 99% identical to the sequence of SEQ ID NO:5.

In the method of the invention, the MERIT40 subunit may be encoded by a nucleic acid sequence that is at least 85%, 90%, 95%, 96%, 97%, 98%, or 99% identical to the sequence of SEQ ID NO:7.

In the method of the invention, the one or more subunits of the one or more BRISC monomers may be labelled with a first detection label from a detection pair and wherein one or more subunits of the one or more BRISC monomers is labelled with a second detection label from the detection pair.

In the method of the invention, the detection pair may comprise a fluorescent detection pair and/or one or a luminescent detection pair.

In the method of the invention, the first detection label may comprise a donor and the second detection label may comprise an acceptor.

In the method of the invention, the detection pair may comprise a Fluorescent resonance energy transfer (FRET) pair or a Bioluminescence resonant energy transfer (BRET) pair.

In the method of the invention, the FRET pair may comprise mClover-m Ruby, CyPet-YPet, EGFP-mCherry, Venus-mCherry, Venus-tdTomato, Venus-mPlum, EBFP2-mEGFP, ECFP-EYFP, Cerulean-Venus, MiCy-mKO, or TFP1-mVenus, optionally mClover3-mRuby3.

In the method of the invention, the BRET pair may comprise Nanoluciferase-mCherry, Nanoluciferase-HaloTag, Nanoluciferase-Venus, Luciferase-GFP, Luciferase-YFP, Luciferase-Venus or LgiT-smBiT.

In the method of the invention, the donor may be attached to the C-terminus of the BRCC45 subunit and/or the acceptor may be attached to the C-terminus of the BRCC36 subunit, optionally wherein the donor may be attached to position 383 of BRCC45 such as L383 and/or the acceptor may be attached to position 316 of BRCC36 such as E316.

In the method of the invention, the acceptor may be attached to the C-terminus of the BRCC45 subunit and/or the donor may be attached to the C-terminus of the BRCC36 subunit, optionally wherein the acceptor may be attached to position 383 of BRCC45 such as L383 and/or the donor may be attached to position 316 of BRCC36 such as E316.

In the method of the invention, the fluorescent detection pair may comprise a split fluorescent molecule, optionally a split GFP molecule or a spyTag catcher pair.

In the method of the invention, one half of the split fluorescent molecule may be attached to the C-terminus of the BRCC45 subunit as the first detection label and the other half of the split fluorescent molecule may be attached to the C-terminus of the BRCC36 subunit as the second detection label.

In the method of the invention, the one or more subunits of BRISC is encoded by a nucleotide sequence may comprise the sequence of SEQ ID NO: 9, SEQ ID NO: 10, SEQ ID NO: 11, SEQ ID NO: 12 or SEQ ID NO: 13.

In the method of the invention, the formation of BRISC dimers can be detected by (i) mass spectrometry and/or (ii) measuring fluorescence signals and/or luminescence signals before and after addition of the compound, wherein for (ii) a relative increase in fluorescence signals and/or luminescence signals indicates an increase in the level of BRISC dimers or an increase in the fluorescence signal of the acceptor together with a decrease in the fluorescence signal of the donor indicates an increase in the level of BRISC dimers.

In the method of the invention, an increase in the level of BRISC dimers of about 10% of BRISC after contacting the population of BRISC monomers with the test compound identifies the test compound as a selective BRISC inhibitor.

The present invention also provides a BRISC dimer comprising:
(i) a first detection label attached to a first BRISC monomer; and
(ii) a second detection label attached to a second BRISC monomer, wherein the first detection label of (i) and the second detection label of (ii) are fused to each other.

In the BRISC dimer of the invention, the first detection label and the second detection label may be fused to each other by an intermediate molecule.

In the BRISC dimer of the invention, the first detection label may be beta1-10 of GFP and the second detection label may be beta 11 of GFP and the intermediate molecule is a Gly-Ser linker.

In the BRISC dimer of the invention, the first detection label may be FRB and the second detection label may be FKBP and the intermediate molecule may be rapamycin.

In the BRISC dimer of the invention, each BRISC monomer may comprise an octameric complex comprising the subunits, Abraxas2, BRCC36, BRCC45 and MERIT40 at a 2:2:2:2 ratio and the BRISC dimer may comprises a 16-meric complex comprising the subunits, Abraxas2, BRCC36, BRCC45 and MERIT40 at a 4:4:4:4 ratio.

In the BRISC dimer of the invention, the first detection label may be attached to the C-terminus of BRCC45 of the first BRISC monomer and the second detection label may be attached to the C-terminus of BRCC36 of a second BRISC monomer.

The present invention also provides the use of a BRISC dimer of the invention to generate cryo-Electron Microscopy (cryo-EM), crystallography, nuclear magnetic resonance and/or X-ray crystallography structures for structure guided drug design.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Embodiments of the invention are further described hereinafter with reference to the accompanying drawings, in which:

FIGS. 1A-1F show the development a high-throughput screen to identify first-in-class DUB inhibitors. FIG. 1A depicts a diagram of a TAMRA-linked IQF di-ubiquitin substrate (left) and BRISC DUB activity against a fluorescent K63-linked substrate (right). FIG. 1B shows the B-score from a high-throughput screen and identification of hit compounds in wells H20, P12. FIG. 1C depicts chemical structures of AT7519 and two isomers with an additional dichlorobenzaldehyde moiety, AP-5-144, JMS-175-2. FIG. 1D shows an IQF assay comparing H20 compound with the two potential isomers, AP-5-144 and JMS-175-2. FIG. 1E shows an IQF assay with JAMM/MPN DUB enzymes; ARISC, and ARISC-RAP80. FIG. 1F shows an IQF assay with JMS-175-2 analogues.

FIGS. 2A-2F show that BRISC inhibitors induce a new BRISC conformation. FIG. 2A shows mass photometry histograms of purified BRISC enzyme in absence of any inhibitor (DMSO). 72% counts correspond to BRISC monomer, 2% BRISC dimer and 19% to a dissociated complex. FIG. 2B shows mass photometry histograms of purified BRISC enzyme mixed with JMS-175-2 and FX-171-C. For BRISC and JMS-175-2, counts are 8% 1:1:1:1 complex, 14% monomer, 69% dimer. BRISC and FX-171-C—13% 1:1:1:1 complex, 22% monomer, 52% dimer. FIG. 2C shows the Representative 2D classes from negative stain electron microscopy of BRISC with different inhibitors. FIG. 2D is the native mass spectrometry spectra of BRISC and BRISC mixed with JMS-175-2 or FX-171-C inhibitor. The three major BRISC species observed are highlighted. FIG. 2E is a table of calculated masses for different BRISC subcomplexes and supercomplexes. FIG. 2F shows mass photometry measurements at increasing inhibitor concentration. Proportion of counts corresponding to dimer are plotted. Data are from three independent experiments.

FIGS. 3A-3F show that inhibitors bind at the interface of two BRISC complexes. FIG. 3A shows the cryo-EM density of a 3.5 Å BRISC-JMS-175-2 structure. BRISC monomers are labelled in grey and salmon. FIG. 3B shows the cryo-EM density map for BRISC-JMS-175-2 structure. BRISC subunits are coloured by corresponding chain and inhibitor density highlighted in red and highlighted in red boxes. FIG. 3C shows the identification of inhibitor density in the BRISC-JMS-175-2 cryoEM map. Panels identify density for two JMS-175-2 molecules corresponding to the dimer structure in FIG. 3B. FIG. 3D shows the density for BRCC36, Abraxas2 and BRCC45 on opposite side of monomer i.e. where there is no density for JMS-175-2. FIGS. 3E and 3F depict the chemical structures of JMS-175-2 and FX-171-C modelled in cryo-EM density shown as mesh. Compounds modelled in State 1 and State 2.

FIGS. 4A-4C show validation of BRISC-Compound interaction site. FIG. 4A depicts a detailed structure of the BRISC dimer composite pocket and individual residues interacting with the compound are labelled. FIG. 4B shows the compound dose response curves measuring wild type BRISC activity and BRISC complexes containing the indicated BRCC36 (top panel), Abraxas2 (middle panel) and BRCC45 (bottom panel) mutants. FIG. 4C shows the SHMT2 dose response curves measuring wild type BRISC activity and BRISC complexes containing the indicated mutants.

FIGS. 5A-5E show validation of hit compounds. FIG. 5A shows dose response curves for hit compounds against BRISC (1 nM), USP2 (100 nM) and Trypsin (125 nM) using the internally-quenched fluorescence di-ubiquitin assay described in FIG. 1A. FIG. 5B shows retesting of purchased H20 hit compound presumed to be AT7519. FIG. 5C shows the UV-vis profile of compound in well H20 and purchased AT7519 compounds from Synkinase and Sellechem. FIG. 5D shows the Liquid-chromatography-mass spectrometry (LC-MS) analysis of H20 compound. FIG. 5E shows the MS fragmentation analyses for H20 compound and a synthesised isomer, AP-5-144.

FIG. 6A shows the chemical structure of BRISC small-molecule inhibitor (FX-171-C). FIG. 6B shows that FX-171-C inhibits longer ubiquitin chains (K63-linked tetraubiquitin; Ub₄)

FIGS. 7A-7D show that cryo-electron microscopy of the apo-BRISC complex reveals a new BRISC conformation. FIG. 7A shows a representative micrograph out of 1,610 movies collected (top), and representative 2D classes from particles picked in crYOLO (bottom). FIG. 7B shows cryoEM processing workflow to generate models for BRISC monomer and dimer. Green indicates selected classes used for 3D refinement. FIG. 7C shows cryoEM density map (45,220 particles) at 6.3 Å resolution with BRISC model (PDB: 6H3C) rigid body fitted. FIG. 7D is a low resolution cryoEM density map of dimeric BRISC (3,244 particles), with two BRISC models fitted.

FIGS. 8A-8F show cryo-electron microscopy processing of a BRISC-JMS-175-2 co-complex. FIG. 8A shows a representative micrograph out of 7,771 movies collected. FIG. 8B shows selected 2D classes (from 100 generated) from particles picked in crYOLO. FIG. 8C shows an image processing workflow. Red asterisks indicate JMS-175-2 binding site. FIG. 8D shows the FSC curves for the final reconstructions used for model building. The final resolution was calculated using the gold-standard FSC cut off at 0.143 frequency. FIG. 8E shows the mask (green) applied during focused refinement of the C1 reconstruction. FIG. 8F shows the density for JMS-175-2 (red) before (top) and after (bottom) focused refinement.

FIGS. 9A-9B show cryo-electron microscopy processing of BRISC-FX-171-C co-complex. FIG. 9A is a representative micrograph out of 9,251 movies collected (top). Selected 2D classes from particles picked in crYOLO (bottom). FIG. 9B shows the image processing workflow.

FIGS. 10A-10B show dimer formation blocks substrate and SHMT2 binding. FIG. 10A shows the K63-linked diUb (dark grey) modelled on the MPN+ domain of BRCC36, based on the AMSH-LP-diUb structure (PDB code 2ZNV). Upon dimer formation (left), the second BRISC monomer sterically clashes with the proximal ubiquitin. FIG. 10B shows the JMS-175-2 binding pocket coloured by molecular lipophilicity potential (MLP) in ChimeraX. Dark gold is the most hydrophobic and dark cyan is the most hydrophilic. SHMT2 (PDB code 6R8F) modelled on the BRISC-JMS-175-2 structure. The SHMT2 α6 helix overlaps with JMS-175-2 binding site.

FIGS. 11A-11B show an analysis of the BLUE binding site. FIG. 11A shows JMS-175-2 binding pocket coloured by molecular lipophilicity potential (MLP) in ChimeraX. Dark gold is the most hydrophobic and dark cyan is the most hydrophilic. FIG. 11B shows quantification of BRISC dimeric species from a mass photometry experiment in the presence of FX-171-C and DMSO.

FIGS. 12A-12C show that BRISC forms asymmetric dimers of octamers. FIG. 12A shows 2D class averages from BRISC cryo-EM datasets DMSO and inhibitor. FIG. 12B shows cryo-EM maps (white surface) and fitted BRISC model. Arrows indicate bound molecular glue. FIG. 12C depicts active and autoinhibited BRISC models with attached molecules such as interchangeable donor and acceptor fluorescent or luminescent labels.

FIGS. 13A-13D show exemplary positions of labels and fusion proteins at selected locations on the C-terminus of BRCC36 and C-terminus of BRCC45. FIG. 13A shows the position of FRET/BRET labels. FIG. 13B shows the position of LgBit and smBiT labels. FIG. 13C shows the position of split GFP (sGFP) whereby beta1-10 can be fused to the C-terminus of BRCC45 and befall of GFP can be fused to the C-terminus of BRCC36 using a Gly-Ser linker. The positions of labels shown in FIGS. 13A-13C can be used to identify selective BRISC inhibitors. FIG. 13D shows the position of FRB and FKBP labels which may, for example, be used to more readily obtain a cryoEM structure. The pairings in FIG. 13C and FIG. 13D can be used to generate a stable BRISC dimer.

FIGS. 14A-14M show exemplary BRISC constructs and the corresponding nucleotide sequences.

### DETAILED DESCRIPTION

BRISC Monomer

The present invention involves BRISC monomers. A BRISC monomer may comprise an octameric complex comprising the subunits, Abraxas2, BRCC36, BRCC45 and MERIT40 or derivatives thereof. Preferably, the BRISC monomer comprises an octameric complex comprising the subunits, Abraxas2, BRCC36, BRCC45 and MERIT40 at a 2:2:2:2 ratio.

The BRISC monomer can be detected by mass spectrometry using methods known in the art.

The Abraxas subunit may be encoded by a nucleotide sequence comprising the sequence of SEQ ID NO: 1. The BRCC36 subunit may be encoded by the nucleotide sequence comprising the sequence of SEQ ID NO: 3. The BRCC45 subunit may be encoded by the nucleotide sequence comprising the sequence of SEQ ID NO: 5. The MERIT40 subunit may be encoded by the nucleotide sequence comprising the sequence of SEQ ID NO: 7. Accordingly, the polypeptide sequence of the Abraxas subunit may comprise the sequence of SEQ ID NO:2. The polypeptide sequence of the BRCC36 subunit may comprise the sequence of SEQ ID NO:4. The polypeptide sequence of the BRCC45 subunit may comprise the sequence of SEQ ID NO:6. The polypeptide sequence of the MERIT40 subunit may comprise the sequence of SEQ ID NO:8.

Alternatively, the Abraxas subunit may be encoded by a nucleotide sequence that is at least 85%, 90%, 95%, 96%, 97%, 98%, 99% identical to the sequence of SEQ ID NO: 1. The BRCC36 subunit may be encoded by a nucleotide sequence that is at least 85%, 90%, 95%, 96%, 97%, 98%, 99% identical to sequence of SEQ ID NO: 3. The BRCC45 subunit may be encoded by a nucleotide sequence that is at least 85%, 90%, 95%, 96%, 97%, 98%, 99% identical to the sequence of SEQ ID NO: 5. The MERIT40 subunit may be encoded by a nucleotide sequence that is at least 85%, 90%, 95%, 96%, 97%, 98%, 99% identical to the sequence of SEQ ID NO: 7. Accordingly, the polypeptide sequence of the Abraxas subunit may comprise an amino acid sequence that is at least 85%, 90%, 95%, 96%, 97%, 98%, 99% identical to sequence of SEQ ID NO:2. The polypeptide sequence of the BRCC36 subunit may comprise an amino acid sequence that is at least 85%, 90%, 95%, 96%, 97%, 98%, 99% identical to the sequence of SEQ ID NO:4. The polypeptide sequence of the BRCC45 subunit may comprise an amino acid sequence that is at least 85%, 90%, 95%, 96%, 97%, 98%, 99% identical to the sequence of SEQ ID NO:6. The polypeptide sequence of the MERIT40 subunit may comprise an amino acid sequence that is at least 85%, 90%, 95%, 96%, 97%, 98%, 99% identical to the sequence of SEQ ID NO:8.

Calculations of sequence homology or identity between sequences are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 82%, 84%, 85%, 86%, 87%, 88%, 89%, 90%, 91%, 92%, 93%, 94%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

A BRISC monomer may be produced by any known means. For example, affinity purification of BRISC monomers may be employed as a means to produce a population of BRISC monomers.

The invention may also involve the use of cells that have been modified to express one or more BRISC monomers. Such cells may be modified to carry an expression vector encoding a BRISC dimer disclosed herein. Such cells typically include eukaryotic cells such as insect cells, for example Spodoptera frugiperda (S19) cells. Such cells may include prokaryotic cells such as bacterial cells, for example E. coli. Such cells may be cultured using routine methods to produce a polypeptide of the invention.

The BRISC monomer may be modified. It may be modified by the attachment of one or more detection labels as disclosed herein. The BRISC monomer may be modified by the attachment of one or more such detection labels to any one or more subunits, Abraxas2, BRCC36, BRCC45 and/or MERIT40. The BRISC monomer may be modified by the attachment of one or more detection labels to the C-terminus of BRCC45 and/or to the C-terminus of BRCC36. The BRISC monomer may be modified by the attachment of a first detection label, for example a donor molecule, disclosed herein to the C-terminus of the BRCC45 subunit and/or the BRISC monomer may be modified by the attachment of a first detection label, for example an acceptor molecule, as disclosed herein to the C-terminus of the BRCC36 subunit. The BRISC monomer may be modified by the attachment of an acceptor molecule disclosed herein to the C-terminus of the BRCC45 subunit and/or the BRISC monomer may be modified by the attachment of a donor molecule disclosed herein to the C-terminus of the BRCC36 subunit. The BRISC monomer may be modified by the attachment of one half of a split fluorescent molecule disclosed herein to the C-terminus of the BRCC45 subunit and the other half of the split fluorescent molecule disclosed herein is attached to the C-terminus of the BRCC36 subunit. The BRISC monomer may be modified by the attachment of FRB (FK506-rapamycin binding) to the C-terminus of the BRCC45 subunit and/or FKBP (FK506-binding protein) to the C-terminus of the BRCC36 subunit. The BRISC monomer may be modified by the attachment of FKBP to the C-terminus of the BRCC45 subunit and/or FRB to the C-terminus of the BRCC36 subunit. The BRISC monomer may be modified at position 383 of BRCC45 such as L383 and/or at position 316 of BRCC36 such as E316 by the attachment of acceptor, donor molecules, and/or detection labels as disclosed herein.

BRISC Dimer

The present invention involves BRISC dimers. A BRISC dimer may comprise a 16-meric complex comprising the subunits, Abraxas2, BRCC36, BRCC45 and MERIT40 or derivatives thereof. Preferably, the BRISC monomer comprises an octameric complex comprising the subunits, Abraxas2, BRCC36, BRCC45 and MERIT40 at a 4:4:4:4 ratio.

The BRISC dimer can be detected by mass spectrometry using methods known in the art. The BRISC dimer may also be detected through the use of detection labels disclosed herein that emit a fluorescent or bioluminescent signal upon formation of the BRISC dimer by two BRISC monomers.

The Abraxas subunit may be encoded by a nucleotide sequence comprising the sequence of SEQ ID NO: 1. The BRCC36 subunit may be encoded by the nucleotide sequence comprising the sequence of SEQ ID NO: 3. The BRCC45 subunit may be encoded by the nucleotide sequence comprising the sequence of SEQ ID NO: 5. The MERIT40 subunit may be encoded by the nucleotide sequence comprising the sequence of SEQ ID NO: 7. Accordingly, the polypeptide sequence of the Abraxas subunit may comprise the sequence of SEQ ID NO:2. The polypeptide sequence of the BRCC36 subunit may comprise the sequence of SEQ ID NO:4. The polypeptide sequence of the BRCC45 subunit may comprise the sequence of SEQ ID NO:6. The polypeptide sequence of the MERIT40 subunit may comprise the sequence of SEQ ID NO:8.

Alternatively, the Abraxas subunit may be encoded by a nucleotide sequence that is at least 85%, 90%, 95%, 96%, 97%, 98%, 99% identical to the sequence of SEQ ID NO: 1. The BRCC36 subunit may be encoded by a nucleotide sequence that is at least 85%, 90%, 95%, 96%, 97%, 98%, 99% identical to sequence of SEQ ID NO: 3. The BRCC45 subunit may be encoded by a nucleotide sequence that is at least 85%, 90%, 95%, 96%, 97%, 98%, 99% identical to the sequence of SEQ ID NO: 5. The MERIT40 subunit may be encoded by a nucleotide sequence that is at least 85%, 90%, 95%, 96%, 97%, 98%, 99% identical to the sequence of SEQ ID NO: 7. Accordingly, the polypeptide sequence of the Abraxas subunit may comprise an amino acid sequence that is at least 85%, 90%, 95%, 96%, 97%, 98%, 99% identical to sequence of SEQ ID NO:2. The polypeptide sequence of the BRCC36 subunit may comprise an amino acid sequence that is at least 85%, 90%, 95%, 96%, 97%, 98%, 99% identical to the sequence of SEQ ID NO:4. The polypeptide sequence of the BRCC45 subunit may comprise an amino acid sequence that is at least 85%, 90%, 95%, 96%, 97%, 98%, 99% identical to the sequence of SEQ ID NO:6. The polypeptide sequence of the MERIT40 subunit may comprise an amino acid sequence that is at least 85%, 90%, 95%, 96%, 97%, 98%, 99% identical to the sequence of SEQ ID NO:8.

Affinity purification of BRISC dimers may be employed as a means to produce a population of BRISC dimers.

The invention may also involve the use of cells that have been modified to express one or more BRISC dimers. Such cells may be modified to carry an expression vector encoding a BRISC dimer disclosed herein. Such cells typically include eukaryotic cells such as insect cells, for example Spodoptera frugiperda (Sf9) cells. Such cells may include prokaryotic cells such as bacterial cells, for example E. coli. Such cells may be cultured using routine methods to produce a polypeptide of the invention.

The BRISC dimer may be modified. It may comprise modified BRISC monomers as disclosed here. For example, the BRISC dimer may comprise modified BRISC monomers as follows: The BRISC monomer may be modified by the attachment of one or more such detection labels to any one or more subunits, Abraxas2, BRCC36, BRCC45 and/or MERIT40. The BRISC monomer may be modified by the attachment of one or more detection labels to the C-terminus of BRCC45 and/or to the C-terminus of BRCC36. The BRISC monomer may be modified by the attachment of a donor molecule disclosed herein to the C-terminus of the BRCC45 subunit and/or the BRISC monomer may be modified by the attachment of an acceptor molecule to the C-terminus of the BRCC36 subunit. The BRISC monomer may be modified by the attachment of an acceptor molecule disclosed herein to the C-terminus of the BRCC45 subunit and/or the BRISC monomer may be modified by the attachment of a donor molecule disclosed herein to the C-terminus of the BRCC36 subunit. The BRISC monomer may be modified by the attachment of one half of a split fluorescent molecule disclosed herein to the C-terminus of the BRCC45 subunit and the other half of the split fluorescent molecule disclosed herein is attached to the C-terminus of the BRCC36 subunit. The BRISC monomer may be modified by the attachment of FRB to the C-terminus of the BRCC45 subunit and/or FKBP to the C-terminus of the BRCC36 subunit. The BRISC monomer may be modified by the attachment of FKBP to the C-terminus of the BRCC45 subunit and/or FRB to the C-terminus of the BRCC36 subunit.

Further, the BRISC dimer may be a stable BRISC dimer. A stable BRISC dimer may be more amenable to sample preparation for crystallography electron microscopy (EM), crystallography or Nuclear Magnetic Resonance (NMR) studies. A stable BRISC dimer may comprise (i) a first detection label attached to a first BRISC monomer; and (ii) a second detection label attached to a second BRISC monomer, wherein the first detection label of (i) and the second detection label of (ii) are fused to each other. In one aspect, the first detection label of (i) and the second detection label of (ii) are fused to each other by an intermediate molecule. In another aspect, the first detection label of (i) is beta1-10 of GFP and the second detection label of (ii) is beta 11 of GFP and the intermediate molecule is a Gly-Ser linker. In a further aspect, the first detection label of (i) FRB and the second detection label of (ii) is FKBP and the intermediate molecule is rapamycin. The first detection label and/or second detection label may be attached to any subunit of BRISC at any position/residue. In a further aspect, the first detection label is attached to the C-terminus of BRCC45 of the first BRISC monomer and the second detection label is attached the C-terminus of BRCC36 of a second BRISC monomer. The first detection label may be attached to the C-terminus of BRCC36 of the first BRISC monomer and the second detection label may be attached the C-terminus of BRCC45 of a second BRISC monomer.

The BRISC dimers disclosed herein may be used to generate cryo-Electron Microscopy (cryo-EM), crystallography, nuclear magnetic resonance and/or X-ray crystallography structures for structure guided drug design.

Population

A population of BRISC monomers may refer to one or more BRISC monomers disclosed herein.

The screening methods disclosed herein involve contacting a population of BRISC monomers with a compound. A population of BRISC monomers may refer to two or more BRISC monomers.

Selective BRISC Inhibitor

The screening methods disclosed herein enable the identification of a selective BRISC inhibitor. A selective BRISC inhibitor as disclosed herein is a compound which causes dimerization of BRISC monomers upon binding to the BRISC monomers. A selective BRISC inhibitor has the ability to inhibit a biological function of a native BRISC deubiquitinating enzyme. Accordingly, the term “inhibitor” is defined in the context of the biological role of BRISC. A selective BRISC inhibitor may specifically interact with (e.g., bind to) BRISC. BRISC biological activity inhibited by a BRISC inhibitor may be associated with the inhibition of Type I interferon receptor (IFNARI) in addition to toll like receptors 4 and 7 (TLR4 and TLR 7).

A chemical or compound that “selectively” inhibits BRISC is a chemical or compound that inhibits the expression of activity of BRISC, but does not inhibit the expression or activity of the DUB types. As used herein, the term “inhibits,” “inhibition,” or “inhibiting” refer to a decrease by any value between 10%) and 90%>, or of any integer value between 30%> and 60%>, or over 100%, or a decrease by 1-fold, 2-fold, 5-fold, 10-fold, or more. Any appropriate chemical or compound can be used according to the presently described methods.

A selective BRISC inhibitor may be a compound which binds to BRISC. A selective BRISC inhibitor may bind to a unique binding site on BRISC which make the inhibitors selective for BRISC unlike other inhibitors previously described in the prior art. Accordingly, a selective BRISC inhibitor may further be a compound which does not bind to other DUBs. It may also be an inhibitor which binds with greater affinity to BRISC than any other DUBs. A selective BRISC inhibitor may bind to one or more residues present in BRISC, for example in the subunits, Abraxas2, BRCC36, BRCC45 and/or MERIT40. For example, the selective BRISC inhibitor may bind to one or more residues present in: the S-loop of BRCC36 S-loop, the X-loop of BRCC36, and/or the Y-loop of Abraxas2. For example, a selective BRISC inhibitor may bind to any one or more the following: T128 of BRCC36, W130 of BRCC36, I158 of BRCC36, L169 of BRCC36, T140 of Abraxas2, R137 of BRCC45, F140 of BRCC45, C245 of BRCC45, and D248 of BRCC45. Selective BRISC inhibitors may also bind to the active site Zn2+ ion, or BRCC36 catalytic site residues E33, H122, H124, S132 D135. A selective BRISC inhibitor may further be a compound that results in dimerization of BRISC as disclosed herein. That is, a selective BRISC inhibitor may result in an increase of the level of BRISC dimers following contacting with a population of BRISC monomers. Inhibitors identified in this manner may be selective for BRISC. The increase in the level of BRISC dimers may be due to the selective BRISC inhibitor compelling/bringing together two BRISC monomers. The BRISC monomer may comprise an octameric complex comprising the subunits, Abraxas2, BRCC36, BRCC45 and MERIT40 at a 2:2:2:2 ratio. The BRISC dimer may comprise a 16-meric complex comprising the subunits, Abraxas2, BRCC36, BRCC45 and MERIT40 at a 4:4:4:4 ratio. Examples of selective BRISC inhibitors may be FX-171-C and JMS-175-2 and derivatives thereof.

Detection Pairs

The present invention may involve the use of one or more detection pairs, comprising a first detection label and a second detection label. One or more subunits of the one or more BRISC monomers may be labelled with a first detection label from a detection pair and one or more subunits of the one or more BRISC monomers may be labelled with a second detection label from a detection pair. In one aspect, BRISC monomers may be labelled with one or more detection labels from a detection pair (e.g., a BRISC monomer may have one or more detection labels attached to it). The label may be covalently or non-covalently conjugated to a BRISC monomer. These detection pairs enable the detection of BRISC dimers. The detection pair is an energy transfer pair comprising a donor and an acceptor. The term “donor” refers to a fluorescent resonance energy transfer (FRET) energy donor, or a bioluminescence energy transfer (BRET) energy donor. The term “acceptor” refers to a fluorescent resonance energy transfer (FRET) energy acceptor or a bioluminescence energy transfer (BRET) energy acceptor and an AlphaScreen acceptor bead.

In these embodiments the level of BRISC dimer formation may be measured determining an energy transfer, wherein one BRISC monomer provides an energy donor, and a further, different, BRISC monomer provides an energy acceptor.

For example, the detection labels of the detection pair may comprise one or more fluorescent reporter molecules and/or one or more luminescent reporter molecules. Preferably, the one or more fluorescent molecules may be mClover, mRuby, CyPet, YPet, EGFP, mCherry, Venus, tdTomato, mPlum, EBFP2, mEGFP, ECFP, EYFP, Cerulean, MiCy, mKO, TFP1, mClover3, or mRuby3. Preferably the one or more luminescent reporter molecules may be nanoluciferase or luciferase. For example, a fluorescent reporter molecule may be encoded by a nucleotide sequence comprising the sequence of SEQ ID NO: 9, SEQ ID NO: 10, SEQ ID NO: 11, SEQ ID NO: 12 or SEQ ID NO: 13.

In one embodiment each member of the detection pair is conjugated to a separate BRISC monomer. Upon BRISC dimerization the members of the detection pair will interact with one another, and thus bring the detection pair into close proximity with one another. Upon exposure to excitation, for example light, the detection pair will generate a signal. In the methods according to the invention, the donor and acceptor are each conjugated to a different BRISC monomer.

The one or more fluorescent detection pair or luminescent detection pair comprise a donor and acceptor pair. Preferably, the donor and acceptor pair comprise a Fluorescent resonance energy transfer (FRET) pair or a Bioluminescence resonant energy transfer (BRET) pair.

Fluorescence resonance energy transfer (FRET) is a distance-dependent physical process by which energy is transferred nonradiatively from an excited molecular fluorophore (the donor) to another fluorophore (the acceptor) by means of intermolecular long-range dipole-dipole coupling. BRET works in a similar manner. It also involves energy transfer between one light-emitting molecule (typically a luciferase) and a light-sensitive molecule (typically a fluorescent protein). Preferably, the donor-acceptor pair is any one of the following mClover-mRuby, CyPet-YPet, EGFP-mCherry, Venus-mCherry, Venus-tdTomato, Venus-mPlum, EBFP2-mEGFP, ECFP-EYFP, Cerulean-Venus, MiCy-mKO, or TFP1-mVenus, mClover3-mRuby3, Nanoluciferase-mCherry, Nanoluciferase-HaloTag, Nanoluciferase-Venus, Luciferase-GFP, Luciferase-YFP, Luciferase-Venus LgiT-smBiT, Nanoluciferase-mCherry, Nanoluciferase-HaloTag, Nanoluciferase-Venus, Luciferase-GFP, Luciferase-YFP, Luciferase-Venus and LgiT-smBiT. The donor and/or acceptor may be attached to any subunit of BRISC at any position/residue. For example, the donor and/or acceptor may be attached to one or more of Abraxas2, BRCC36, BRCC45 and MERIT40. The C-terminus of the BRCC45 subunit and/or the acceptor may be attached to the C-terminus of the BRCC36 subunit, optionally wherein the donor is attached to position 383 of BRCC45 such as L383 and/or the acceptor is attached to residue 316 of BRCC36 such as E316. The acceptor is attached to the C-terminus of the BRCC45 subunit and/or the donor is attached to the C-terminus of the BRCC36 subunit, optionally wherein the acceptor is attached to residue 383 of BRCC45 such as L383 and/or the donor is attached to residue 316 of BRCC36 such as E316.

The invention refers to certain positions of proteins. By “position” as used herein is meant a location in the sequence of a protein. Corresponding positions are determined alignment with other parent sequences. By “residue” as used herein is meant a position in a protein and its associated amino acid identity. For example, reference to positions in the BRCC45 protein is sequential, with residue 1 corresponding to amino acid 1 in SEQ ID NO: 6 and residue 383 corresponding to amino acid 383 in SEQ ID NO:6. Similarly, reference to positions in the BRCC36 protein is sequential, with residue 1 corresponding to amino acid 1 in SEQ ID NO:4 and residue 316 corresponding to amino acid 316 in SEQ ID NO:4.

FRET detection pair refers to a pair of fluorophores consisting of a donor fluorescent compound and an acceptor compound. When the donor and acceptor are in proximity to one another and when they are excited at the excitation wavelength of the donor fluorescent compound, these compounds emit a FRET signal and the energy transfer detected is fluorescence resonance energy transfer. It is known that, in order for two fluorescent compounds to be FRET partners, the emission spectrum of the donor fluorescent compound must partially overlap the excitation spectrum of the acceptor compound. The preferred FRET-partner pairs are those for which the value RO (Förster distance, distance at which energy transfer is 50% efficient) is greater than or equal to 30 Å.

A FRET signal refers to any measurable signal representative of FRET between a donor fluorescent compound and an acceptor compound. A FRET signal can therefore be a variation in the intensity or in the lifetime of luminescence of the donor fluorescent compound or of the acceptor compound when the latter is fluorescent.

Bioluminescence resonance energy transfer (BRET) or example, in which the donor compound is, e.g., luciferase, may be used in place of FRET. Accordingly in one embodiment the donor and acceptor are a bioluminescence energy transfer (BRET) donor and acceptor and the energy transfer detected is bioluminescence energy transfer (BRET). The BRET assay technology is based on the efficient Resonance Energy Transfer (RET) between a bioluminescent donor moiety and a fluorescent acceptor moiety. BRET is a naturally occurring phenomenon and differs from FRET in that it uses a luciferase as the donor. In one embodiment a luciferase (Rluc) isolated from the sea pansy Renilla reniformis and a coelenterazine substrate named DeepBlueC (DBC) are used as the donor. In the presence of oxygen, Rluc catalyzes the transformation of DBC into coelenteramide with concomitant light emission peaking at 395 nm (blue light). When a suitable acceptor is in close proximity, the blue light energy is captured by RET. In one embodiment the acceptor in BRET is a GFP variant (GFP2) that is engineered to maximally absorb the energy emitted by the Rluc/DBC reaction. Excitation of GFP2 by RET results in an emission of green light at 510 nm. Energy transfer efficiencies between Rluc/DBC and GFP2 are determined ratiometrically by dividing the acceptor emission intensity by the donor emission intensity. This ratiometric measurement is referred to as the BRET signal and reflects the proximity of Rluc to GFP. In another embodiment Rluc is used as the donor, the derivative of coelenterazine as its substrate and a yellow fluorescent protein (YFP) as the acceptor.

Preferably, the detection pair is a fluorescent detection pair comprising a split fluorescent molecule. A split fluorescent molecule may be a molecule which fluoresces when both halves of the split fluorescent molecule come together, such as when the two (non-fluorescing) halves spontaneously associate to form a functional fluorescent protein. A split fluorescent molecule may spontaneously associate when one BRISC monomer binds to a second BRISC monomer to form a BRISC dimer thus producing a fluorescent signal that can be detected as disclosed herein. A split fluorescent molecule may be any that is known in the art, for example, as used to assess protein-protein interactions, such as split GFP or a spyTag catcher pair. One half of the split fluorescent molecule may be attached to the C-terminus of the BRCC45 subunit as the first detection label and the other half of the split fluorescent molecule may be attached to the C-terminus of the BRCC36 subunit as the second detection label. Preferably, the split fluorescent molecule comprises the polypeptide, GFP1-10/beta 1-10 (residues 1-214 of GFP) and the polypeptide, GFP11/beta 11 (residues 215-230 of GFP).

The one or more detection labels may also be one or more molecules that allow for affinity purification of BRISC monomers or BRISC dimers such as those known in the art including Histidine affinity tag (His tag), flag tag, and myc tag.

The BRISC monomer comprising one or more detection labels may accordingly be encoded by a nucleotide sequence comprising the sequence of SEQ ID NO: 9, SEQ ID NO: 10, SEQ ID NO: 11, SEQ ID NO: 12 or SEQ ID NO: 13.

Intermediate Molecule

The present invention may involve an intermediate molecule. A BRISC dimer disclosed herein may comprise (i) a first detection label attached to a first BRISC monomer; and a second detection label attached to a second BRISC monomer, wherein the first detection level of (i) and the second detection label of (ii) are fused together. In one aspect, the first detection label and the second detection label are fused to each other by an intermediate molecule. The intermediate molecule may be any molecule which bridges the first detection label of a first BRISC monomer to the second detection label of a second BRISC monomer to form a BRISC dimer. The intermediate molecule may be a Gly-Ser linker in the case that the first detection label of (i) is beta1-10 of GFP and the second detection label of (ii) is beta 11 of GFP. The intermediate molecule may be rapamycin in the case that the first detection label of (i) is FRB and the second detection label of (ii) is FKBP and the intermediate molecule is rapamycin. In such case, rapamycin can be introduced to a first BRISC monomer comprising FRB as first detection label and a second BRISC monomer comprising FKBP as second detection label to induce BRISC dimer formation caused by rapamycin binding with high affinity to FRB and FKBP. The first detection label and/or second detection label may be attached to any subunit of BRISC at any position/residue. The first detection label may be attached to the C-terminus of BRCC45 of the first BRISC monomer and the second detection label may be attached the C-terminus of BRCC36 of a second BRISC monomer. The first detection label may be attached to the C-terminus of BRCC36 of the first BRISC monomer and the second detection label may be attached the C-terminus of BRCC45 of a second BRISC monomer. The first detection label may be attached to position 383 of BRCC45 such as L383 or attached to position 316 of BRCC36 such as E316. The second detection label may be attached to position 383 of BRCC45 such as L383 or attached to position 316 of BRCC36 such as E316. Accordingly, the first detection label may be attached to position 383 of BRCC45 such as L383 and the second detection label may be attached to position to position 316 of BRCC36 such as E316. Similarly, the first detection label may be attached to position 316 of BRCC36 such as E316 and the second detection label may be attached to position 383 of BRCC45 such as L383.

Measuring Increase

The present invention involves measuring the level of BRISC dimers before and after contacting a population of BRISC monomers with a test compound. In one aspect, the BRISC dimers may be detected by (i) mass spectrometry and/or (ii) measuring fluorescence signals and/or luminescence signals, carried out before and after addition of the test compound.

The term “increased” or “increase” as used herein generally means a difference between the level of BRISC dimer formation in a population of BRISC monomers contacted with a test compound and the level of BRISC Dimer formation in a reference population of untreated BRISC monomers. In some embodiments the level of dimer formation in the population of BRISC monomers contacted with a test compound is at least about 3% greater than the reference population, for example at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90% greater than the reference population. For instance, at least about 10% may be selected as this may be approximately 2-fold higher than the natural/background state of BRISC dimers which have not been contacted with a BRISC inhibitor. An increase in the level of BRISC dimers of about 10% of BRISC after contacting the population of BRISC monomers with the test compound may identify the test compound as a selective BRISC inhibitor

Formation of BRISC dimers (from BRISC monomers) may be detected by mass spectrometry by monitoring the shift of BRISC monomers (octameric complex comprising the subunits, Abraxas2, BRCC36, BRCC45 and MERIT40 at a 2:2:2:2 ratio) to BRISC dimers (16-meric complex comprising the subunits, Abraxas2, BRCC36, BRCC45 and MERIT40 at a 4:4:4:4 ratio). An increase in BRISC dimer following contacting with compound compared with before addition of the test compound may identify the compound as a selective BRISC inhibitor.

A relative increase in fluorescence signals and/or luminescence signals may indicate an increase in the formation of BRISC dimers. In turn, an increase in BRISC dimers may identify a compound as a selective BRISC inhibitor. Detection of fluorescence signals and/or luminescence signals may be carried out using any method known in the art. For example, in the case that a split GFP fluorescent molecule is used as an detection label such (or a fluorescent detection pair), a presence of a GFP signal indicates the presence of a dimer. As a further example, in the case that FRET and BRET are utilised, the formation of dimers can be inferred by measuring the fluorescence intensity ratio of the acceptor and the donor fluorophores and observing the increase of the fluorescence intensity in the acceptor channel with the simultaneous decrease of the intensity in the donor channel. An increase of the fluorescence intensity in the acceptor channel with the simultaneous decrease of the intensity in the donor channel indicates BRISC dimer formation. An exemplary buffer for use in such an assay may comprise 25 mM HEPES, pH 7.0, 100 mM NaCl, 1 mM DTT, 0.1 mg/ml BSA, 0.005% TWEEN-20.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The amino acid sequence of a BRISC dimer or BRISC monomer as described herein may comprises the amino acid sequence of the whole or a portion of a reference sequence defined by SEQ ID NO in which modifications, such as amino acid additions, deletions or substitutions are made relative to the reference sequence or portion thereof. The modifications may be conservative or non-conservative amino acid substitutions. Where there are multiple modifications in a single polypeptide, the modifications in the polypeptide sequence may be a combination of conservative and non-conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in the Table below.

TABLE 1
Chemical properties of amino acids

| | | | |
|---|---|---|---|
| Ala (A) | aliphatic, hydrophobic, neutral | Met (M) | hydrophobic, neutral |
| Cys (C) | polar, hydrophobic, neutral | Asn (N) | polar, hydrophilic, neutral |
| Asp (D) | polar, hydrophilic, charged (−) | Pro (P) | hydrophobic, neutral |
| Glu (E) | polar, hydrophilic, charged (−) | Gln (Q) | polar, hydrophilic, neutral |
| Phe (F) | aromatic, hydrophobic, neutral | Arg (R) | polar, hydrophilic, charged |
| | | | (+) |
| Gly (G) | aliphatic, neutral | Ser (S) | polar, hydrophilic, neutral |
| His (H) | aromatic, polar, hydrophilic, | Thr (T) | polar, hydrophilic, neutral |
| | charged (+) | | |
| Ile (I) | aliphatic, hydrophobic, neutral | Val (V) | aliphatic, hydrophobic, |
| | | | neutral |
| Lys (K) | polar, hydrophilic, charged(+) | Trp (W) | aromatic, hydrophobic, |
| | | | neutral |
| Leu (L) | aliphatic, hydrophobic, neutral | Tyr (Y) | aromatic, polar, hydrophobic |

Where amino acids have similar polarity, this can be determined by reference to the hydropathy scale for amino acid side chains in the Table below.

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One of skill in the art may determine whether any fragment or variant of BRISC possesses enzymatic activity by performing a functional test for activity as described further herein.

All references, patents and patent applications disclosed herein are incorporated by reference with respect to the subject matter for which each is cited, which in some cases may encompass the entirety of the document.

The methods and uses disclosed herein may be conducted in vivo or in vitro. For example, the methods disclosed herein may be cell based. Accordingly, the method of identifying a selective BRISC inhibitor may be a cell-based screen using cells as disclosed herein.

### EXAMPLES

### Example 1—Materials and Methods

Expression and Purification of BRISC Complex

The four-subunit human BRISC (full-length (FL) and MERIT40ΔN, Abraxas2ΔC) complex was cloned using the MultiBac system and co-expressed in Spodoptera frugiperda (Sf9) insect cells³². BRISC mutants were cloned into pFastBac vectors in the Bac-to-Bac system (ThermoFisher), baculoviruses generated in Sf9 cells and used for co-infection of Trichoplusia ni (Tni) cells. All BRISC complexes were purified as previously described^{12,13}.

DUB Activity Assays (IQF)

BRISC complexes were tested for activity in DUB reaction buffer containing 50 mM HEPES-NaOH pH 7.0, 100 mM NaCl, 0.1 mg/mL BSA, 1 mM DTT, and 0.003% Brij-35. DUB activity was measured at a final concentration of 1 nM or 5 nM for less active mutants. Inhibitors were assayed at concentrations up to 200 μM. 20 μL enzyme reactions were carried out in 384-well black flat-bottom low flange plates (Corning; 35373). DUB activity was measured using internally quenched fluorescent (IQF) K63-linked diUb (Lifesensors, catalogue number: DU6303) at 50 or 100 nM. Cleaved diUb was monitored by measuring fluorescence intensity (Ex. 540 nm, Em. 580 nm; dichroic mirror 560 nm). Fluorescence intensity was measured every minute for 30 minutes at 30° C. Fluorescence intensity units for the first 10 minutes were plotted against time to generate a linear reaction progress curve, where the initial velocity (v0) corresponds to the gradient of the curve. IC50 values were calculated using the GraphPad Prism (v8.0) built-in dose-response equation for inhibitor concentration vs. response (variable slope).

Mass Photometry

Microscope coverslips were prepared as previously described³³. All mass photometry experiments were performed using a One^{MP} mass photometer (Refeyn Ltd, Oxford, UK). For preliminary BRISC-inhibitor measurements, 1 μM BRISC(FL) was mixed with either DMSO or inhibitor at 330 μM and incubated for 15 minutes on ice. Prior to MP measurement, the BRISC-inhibitor mix was diluted in gel filtration buffer (25 mM HEPES, 150 mM NaCl, 1 mM TCEP) for a final concentration of BRISC at 10 nM (0.05% DMSO). 12 μL of this dilution was used for the final MP measurement, following autofocus stabilisation. For EC₅₀ measurements with JMS-175-2, FX-171-C and AP-3-60, 2-fold dilutions of inhibitor in 100% DMSO generated a dilution series with concentrations of inhibitor from 800 μM to 0 μM. 0.5 μL inhibitor was mixed with 19.5 μL 50 nM BRISCΔNΔC (2.5% DMSO) and incubated at room temperature for 15 minutes. The BRISC-inhibitor mix was used directly for MP measurement using buffer-free autofocus stabilisation. Movies were recorded for 60 seconds using AcquireMP (Refeyn, UK), and were processed using DiscoverMP (Refeyn, UK). Mass photometry image processing has been previously described³³. Briefly, contrast-to-mass (C2M) calibration was performed using protein standards (66-669 kDa) diluted in gel filtration buffer. The output from each individual movie resulted in a list of particle contrasts which were converted to mass used the C2M calibration. The mass distribution from each run is in a histogram, where count refers to each landing event and a Gaussian sum is fitted to the data. The relative amount of each species is calculated as the area of each Gaussian, where a refers to the standard deviation of the fitted Gaussian. Dimer fraction refers to the percentage of the counts which corresponded to the 564 kDa BRISCΔNΔC dimer complex. Curves were fitted and EC50 values were calculated using the GraphPad Prism (v8.0) built-in dose-response equation for concentration of agonist vs. response (variable slope).

Negative Stain Electron Microscopy—Grid Preparation, Data Collection and Image Processing

BRISC(FL) was mixed with inhibitor (or DMSO) on ice for 30 minutes at a final concentration of 1 μM BRISC and 100 μM inhibitor. The BRISC-inhibitor mix was diluted in gel filtration buffer to a final concentration of 0.014 mg/mL (0.5% DMSO). Sample was immediately loaded onto carbon-coated copper grids (Formvar/Carbon 300 mesh Cu, Agar Scientific). Grids were glow discharged for 30 seconds, at 10 mA, and 0.39 mBar pressure (PELCO easiGlow, Ted Pella). Grids were incubated for 1 minute with 7 μL sample, washed three times with H₂O, stained twice with 2% w/v uranyl acetate for a total of 30 seconds. Excess liquid was removed by blotting with filter paper. Data was collected using an FEI Tecnai F20 microscope at 200 KeV, fitted with an FEI CETA (CMOS CCD) camera. RELION 3.0 and 3.1 were used for processing of negative stain EM data^{34,35}. Approximately, 2000 particles were manually picked and extracted with a box size of 128 Å. These particles were used for reference-free 2D class averaging to generate 2D templates for autopicking. The parameters for autopicking were optimised and between 5000-10000 particles were extracted. Two rounds of 2D classification were used to remove junk particles and assess the stoichiometry of the BRISC complex.

Cryo-Electron Microscopy Grid Preparation and Data Collection

BRISCΔNΔC at 0.3 mg/mL (2 μM) was mixed with JMS-175-2 or FX-171-C at 200 μM in buffer (25 mM HEPES pH 7.5, 150 mM NaCl, 1 mM TCEP) for 30 minutes on ice. BRISC(FL) was loaded directly onto grids at 0.4 mg/mL. Grids were glow-discharged using the GloQube (Quorum) for 30 seconds at 40 mA. An FEI Vitrobot IV (ThermoFisher) was equilibrated to 4° C. at 100% humidity. 3 μL of sample were loaded onto Quantifoil R1.2/1.3 300 mesh copper grids. The grids were then blotted (blot force 3, for 4 seconds) and plunged into liquid ethane cooled by liquid nitrogen for vitrification. Micrograph movies were collected on a Titan Krios microscope (ThermoFisher) at 300 keV. The BRISC(FL) dataset was collected on an FEI Falcon III direct electron detector in integrating mode using a final electron dose of 88 e⁻/Å^{2 36}. The total 75000× magnification was applied and a final calibrated object sampling of 1.065 Å/pixel. 1,610 movies were collected using EPU automated acquisition software. Each exposure movie was 1.5 s, collected over 59 fractions with a dose of 1.49 e⁻/Å² per fraction. One exposure was taken per hole and the defocus values ranged from −1.6 μm to −3.1 μm.

The BRISC-JMS-175-2 (7,771 movies) and BRISC-FX-171-C (9,251) datasets were collected on an FEI Falcon 4 direct electron detector (ThermoFisher). Movies were collected using EPU automated acquisition software in integrating mode with a pixel size of 0.86 Å. The defocus ranged from −1.7 μm to −3.1 μm and the total electron exposure was 0.99 e⁻/Å². Detailed information in Table 3.

TABLE 3
Cryo-EM data collection, refinement and validation statistics

| Data collection and processing | BRISC(FL) | BRISCΔNΔC + JMS-175-2 | BRISCΔNΔC + FX-171-C | Refinement |
|---|---|---|---|---|
| Detector | Falcon [symbol] | Falcon 4 | Falcon 4 | Initial model used (PDB code) |
| | (integrating mode) | (counting mode) | (counting mode) | Model resolution (Å) |
| Magnification | 75,000 × | 96,000 × | 96,000 × | Model resolution range (Å) |
| Voltage ( [symbol] V) | 300 | 300 | 300 | Map sharpening B factor (Å²) |
| Electron exposure ( [symbol] ²) | 1.49 | 0.99 | | Model composition |
| Defocus range (μM) | 1.7 to 3.1 | 1.7 to 3.1 | 1.7 to 3.1 | Non-hydrogen atoms |
| Pixel size (Å) | 1.065 | 0.8 [symbol] | 0.86 | Protein residues |
| Symmetry imposed | C1 | C1, C2 | C1, C2 | B Facter (Å²) |
| Movies collected | 1,610 | 7,7 [symbol] | 9,251 | R.m.s deviations |
| Initial particle images (no.) | 282,442 | 1,616.4 [symbol] 7 | 1,040,845 | Bond lengths (Å) |
| Final particle images (no.) | 38,036 (momomer) | 371,672 | 235,18 [symbol] | Bond angles (°) |
| | 3,244 (dimer) | | | Validation |
| Map resolution (Å) | 6.3 | 3.4 | 4.1 | MolProbity score |
| FSC threshold 0.143 | | | | Clas [symbol] score |
| Map resolution range (Å) | | | | Poor rotamers (%) |
| | | | | Ramachandran plot |
| | | | | Favoured (%) |
| | | | | Allowed (%) |
| | | | | Disallowed (%) |

[symbol]

indicates data missing or illegible when filed

Image Processing

Image processing was carried out using RELION (v.3.0 and v.3.1)^{34,35}. Drift correction was performed using MotionCor2³⁷ and real-time contrast transfer functions were estimated using gCTF³⁸ on-the-fly³⁹.

For the BRISCFL dataset, particles were picked using crYOLO (v.1.3.5)⁴⁰ using a model trained on 10 micrographs. Particles coordinates were imported into RELION 3.0 and 282,442 particles were extracted with a box size of 350 pixels. Particles were subjected to two rounds of reference-free 2D classification in both RELION and cryoSPARC (v3.3.1). For the apo-BRISC structure, classes corresponding to monomeric BRISC (117,891 particles) were selected for cryoSPARC ab initio reconstruction. The best class (38,036 particles) was selected and subjected to homogeneous refinement followed by non-uniform refinement with defocus and global CTF refinement applied. The resulting map is resolved to 6.3 Å, and a BRISC structure with SHMT2 removed (PDB: 6H3C) were rigid body fitted using Chimera (v1.12)⁴¹ and visualised using ChimeraX (v1.2.3)⁴². To process the new BRISC dimer conformation, Relion 2D classes (6,344 particles) corresponding to a BRISC dimer were selected to generate a reference model for 3D classification. Two classes were selected (3,244 particles) for 3D refinement. BRISC structures with SHMT2 removed (PDB: 6H3C) were rigid body fitted using UCSF Chimera (v1.12)⁴¹ and visualised using ChimeraX (v1.2.3).

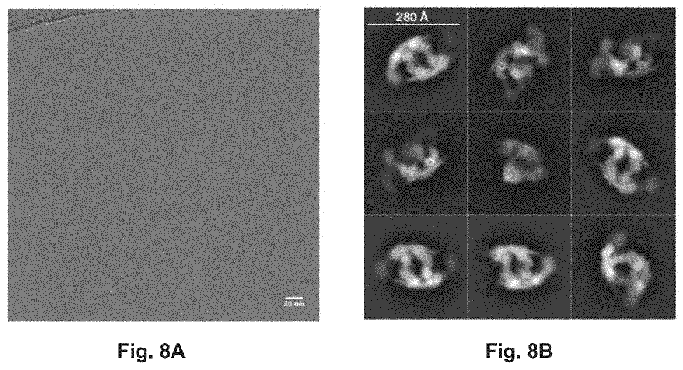

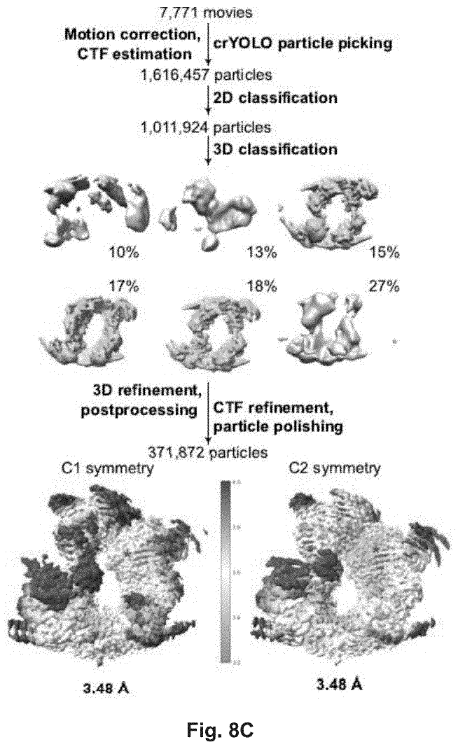

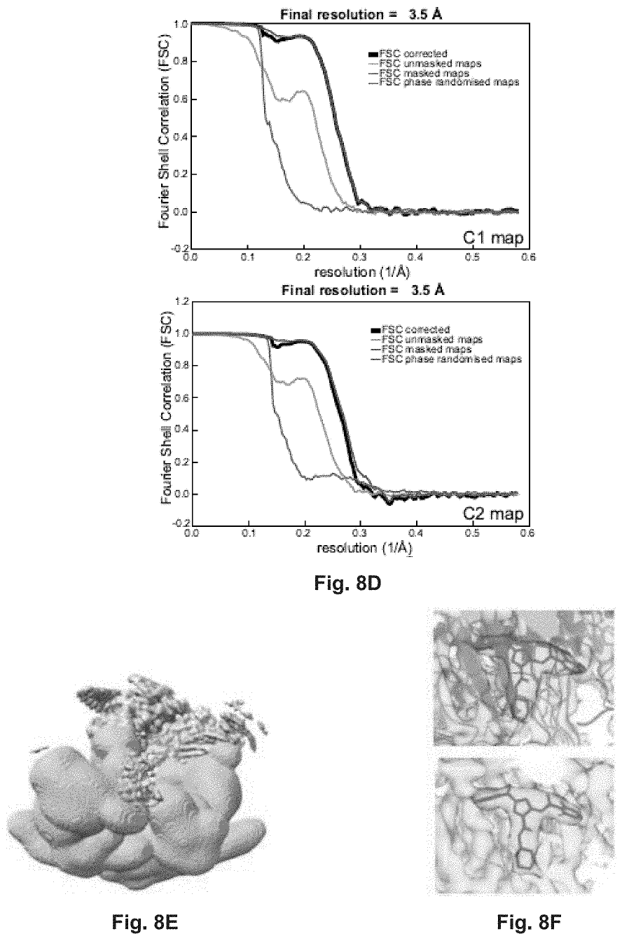

A schematic (FIG. 8) details the data processing pipeline for the BRISCΔNΔC-JMS-175-2 dataset. In summary, particle picking was performed using crYOLO (v.1.6.1)⁴⁰. A model was trained from manually picking 14 micrographs. The trained model picked 1,616,457 particles, for which the coordinates were imported into RELION (v3.1.1) for extraction with 2× binning and a box size of 176 pixels. Two rounds of reference-free 2D classification was used to remove junk particles. A reference model from a previous BRISC-JMS-175-2 dataset was applied during 3D classification of 1,011,924 particles with no symmetry applied. 371,872 particles were selected from 3 classes and re-extracted with a box size of 352 pixels. After 3D refinement and post-processing, a reconstruction of a BRISC dimer complex was achieved at 3.98 Å. Iterative rounds of per-particle contrast function refinement and Bayesian polishing produced an improved final map at 3.48 Å. C2 symmetry was also applied to this map during refinement, and generated an additional map at 3.48 Å. To further improve the density around the small-molecule binding site, a mask was applied during 3D refinement, encompassing only the better resolved half of the C1 map (FIG. 8E). This improved the density for “half” of the structure, resulting in a 3.3 Å map.

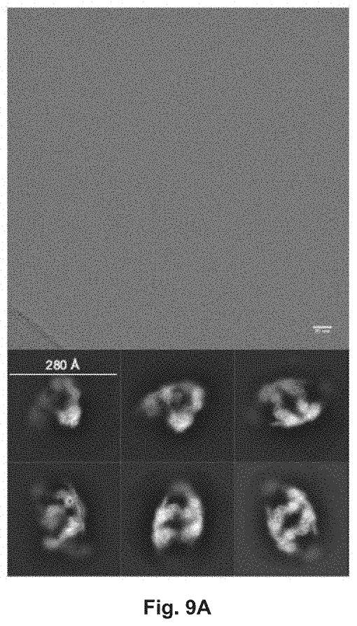

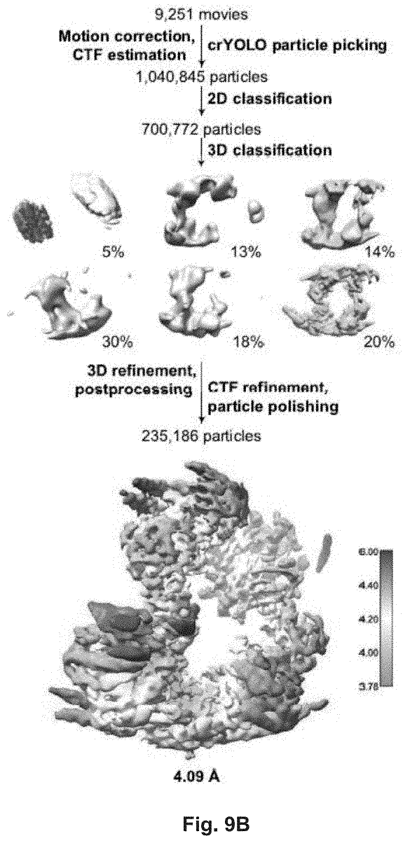

Data processing for the BRISCΔNΔC-FX-171-C dataset is outlined in FIG. 9. Briefly, a model was trained using crYOLO (v.1.6.1)⁴⁰ using particles picked from 10 micrographs, and this model was used to pick 1,040,845 particles which were imported and extracted using RELION (v3.1.1) Particles were binned by 2 and extracted with a box size of 176 pixels. Particles were subjected one round of reference-free 2D classification. A BRISC-JMS-175-2 map was low-pass filtered and used as an initial model for 3D classification with no symmetry applied. The two best classes (235,186 particles) were selected for 3D refinement, post-processing and three rounds of particle polishing and CTF refinement, resulting in a final map at 4.09 Å. Final resolutions were determined using the gold-standard Fourier shell correlation criterion (FSC=0.143). Local resolution estimation was carried out using the RELION local resolution feature.

Model Building and Refinement

A preliminary model of apo-BRISC was acquired from our previous BRISC-SHMT2 cryo-EM structure with SHMT2 structure removed (PDB: 6R8F)¹³. This model was duplicated and two BRISC monomers were rigid-body fitted into the cryoEM density using UCSF Chimera⁴¹. This preliminary model was then subjected to iterative rounds of manual building in COOT⁴³ and refined against the C1 and C2 maps using Phenix real-space refinement (v1.14)⁴⁴. Small-molecule chemical structures were generated in ChemDraw (PerkinElmer, US), and PDB and CIF files were created using the PRODRG2 server⁴⁵. Small-molecules were fit into the density manually using UCSF Chimera, and using COOT Real Space Refine.

Native Mass Spectrometry

BRISC(FL) at 10 μM was mixed with 1 mM inhibitor (JMS-175-2, FX-171-C) or DMSO (2.5%) and incubated on ice for 30 minutes. Samples were buffer exchanged into 500 mM ammonium acetate using Zeba Spin 7K MWCO desalting columns (ThermoFisher). Gold and palladium coated nanospray tips were prepared in-house and mounted on the nano-electrospray ionisation source. Mass spectra were acquired on a Q Exactive Ultra-High Mass Range Hybrid Quadrupole-Orbitrap mass spectrometer (Thermo Fisher), optimised for mass resolution at high m/z ratios. Data were visualised and processed using Mass Lynx (v4.1) and UniDec (v2.7.1).

TABLE 4
HDX-MS Data summary table

| Data Set | BRISC + DMSO | BRISC + FX-171-C | BRISC + AP-3-60 |
|---|---|---|---|
| HDX reaction details | 50 mM potassium phosphate, 200 | 50 mM potassium phosphate, 200 | 50 mM potassium phosphate, 200 |
| | mM NaCl, pD 7.5 [symbol] 95% D2O [symbol] 4° C. | mM NaCl pD 7.5 [symbol] 95% D2O [symbol] 4° C. | mM NaCl pD 7.5 [symbol] 95% D2O, 4° C. |
| HDX time course (min) | 0, 0.5, 1, 10, 60 | | |
| HDX control samples | | | |
| Back-exchange | | | |
| Number of peptides | BRCC36: 110 | BRCC36: 111 | BRCC3 [symbol] : 111 |
| | Abraxas2ΔC: 71 | Abraxas2ΔC: 71 | Abraxas2ΔC: 71 |
| | BRCC45: 110 | BRCC45: 109 | BRCC45: 110 |
| | MERIT40Δn: [symbol] 9 | MERIT40Δn: 09 | MERIT40Δn: [symbol] |
| Sequence coverage (%) | BRCC36: 94.30 | BRCC36: 94.30 | BRCC36: 94.30 |
| | Abraxas2ΔC: 86.8 [symbol] | Abraxas2ΔC: 86.8 [symbol] | Abraxas2ΔC: 86. [symbol] 9 |
| | BRCC45: [symbol] .37 | BRCC45: [symbol] .37 | BRCC45: 88.37 |
| | MERIT40Δn: 92.66 | MERIT40Δn: 92.66 | MERIT40Δn: 92.66 |
| Average peptide length/ | BRCC36: [symbol] .87/3.64 | BRCC36: 9.88/3.6 [symbol] | BRCC36: 9.88/3.68 |
| Redundancy | Abraxas2ΔC: 11.76/3. [symbol] 0 | Abraxas2ΔC: 11.76/3. [symbol] 0 | Abraxas2ΔC: 11.76/3. [symbol] 0 |
| | BRCC45: 9.99/3.21 | BRCC45: 10.00/3.19 | BRCC45: 9.99/3.21 |
| | MERIT40Δn: 9.21/3.42 | MERIT40Δn: 9.21/3.42 | MERIT40Δn: 9.21/3.42 |
| Replicates (biological or | 3 | 3 | 3 |
| technical) | | | |
| Repeatability (average SD) | BRCC36: 0.1155 | BRCC36: 0.0 [symbol] 7 [symbol] | BRCC36: 0.054 [symbol] |
| | Abraxas2ΔC: 0.1155 | Abraxas2ΔC: 0.0 [symbol] 23 | Abraxas2ΔC: 0.0 [symbol] 3 [symbol] |
| | BRCC45: 0.08 [symbol] 1 | BRCC45: 0.0620 | BRCC45: 0.0595 |
| | MERIT40Δn: 0.0 [symbol] 27 | MERIT40Δn: 0.0584 | MERIT40Δn: 0 [symbol] 0.517 |
| Significant differences in HDX | | | |
| (delta HDX > X [symbol] ) | | | |

[symbol]

indicates data missing or illegible when filed

### Example 2—Design of a High-Throughput Screen to Identify Specific BRISC Small-Molecule Inhibitors

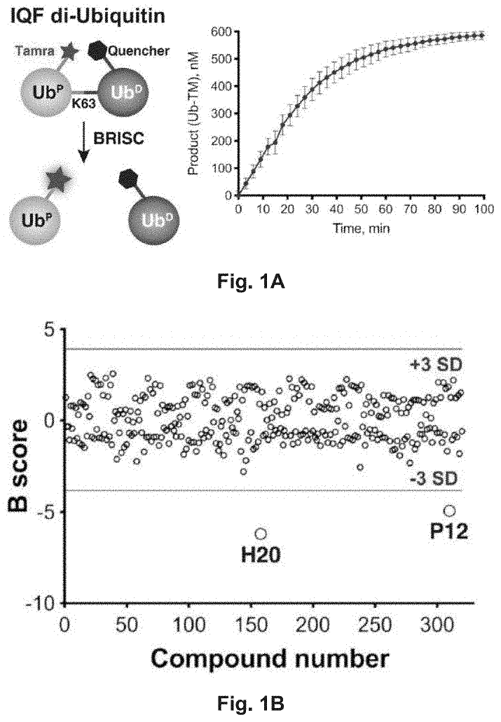

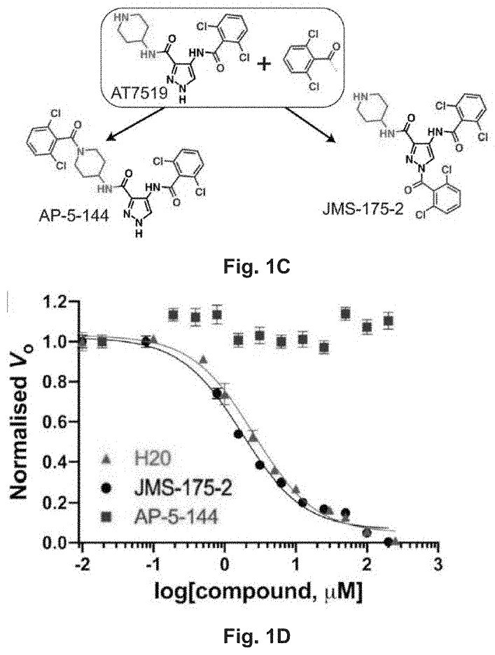

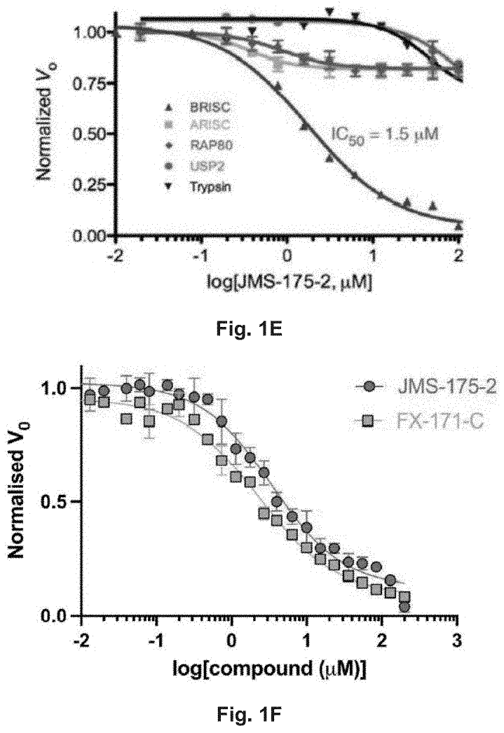

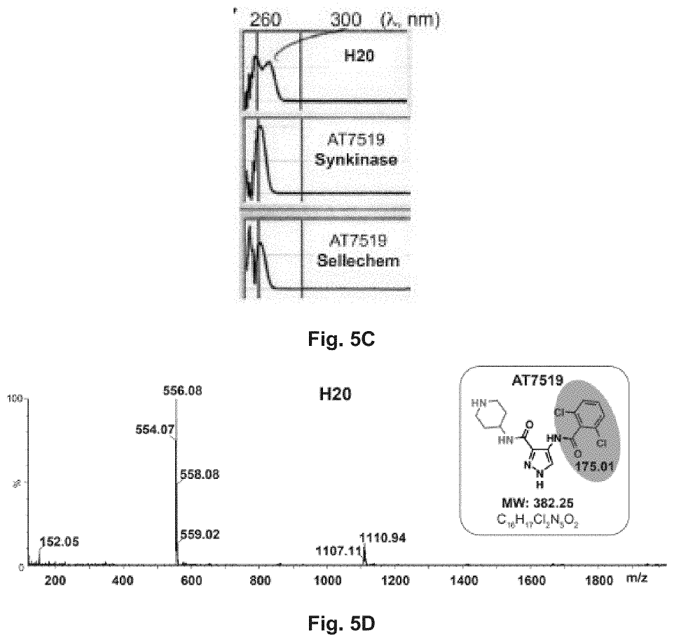

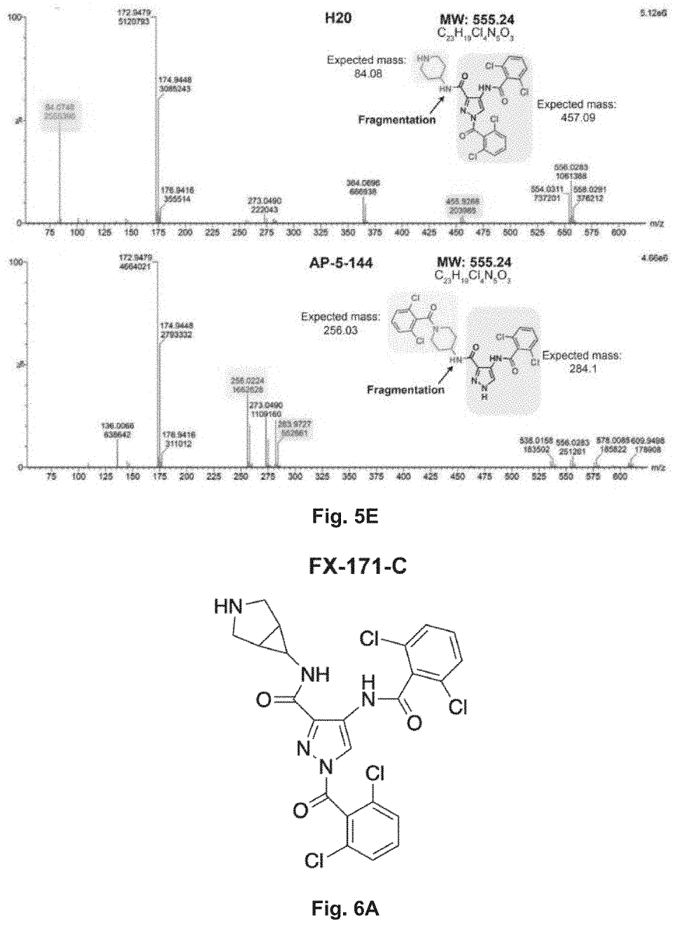

A high throughput biochemical screen was designed to identify inhibitors of BRISC, based on the ability of small molecules to inhibit BRISC cleavage of a K63-linked di-ubiquitin substrate. The di-ubiquitin substrate is designed with a fluorescent TAMRA dye on the proximal ubiquitin moiety and fluorescence quencher on the distal ubiquitin molecule (FIG. 1A). Upon isopeptidase bond cleavage, fluorescence is detected, enabling continuous fluorescent readout of deubiquitylase activity over time (FIG. 1A). The assay conditions were optimised and used to test an in-house compound library comprising of ˜300 published and custom-made kinase inhibitors. From this screen, two hits were identified, compounds AT7519 (well H20) and YM201636 (well P12) (FIG. 1B). The specificity of these compounds was assessed against the broad-spectrum DUB ubiquitin-specific protease-2 (USP2) and the serine protease trypsin which cleave K63-Ub substrate under the same conditions. YM201636 (well P12) inhibited BRISC, trypsin and USP2 suggesting this is a non-specific hit, whilst what we presumed to be compound AT7519 (well H20) showed specific inhibition of BRISC DUB activity (FIG. 5A). To further validate the hit compound in well H₂0, AT7519 from two commercial vendors was used, yet curiously, neither were found to inhibit BRISC DUB activity in the IQF assay (FIG. 5B). UV-Vis spectroscopy analyses of the compound in well H20 used in the screen compared to the Synkinase and Sellechem AT7519 compounds identified an additional peak in the H20 spectra (FIG. 5C). Liquid chromatography-mass spectrometry (LC-MS) revealed the H20 compound had an increased mass, corresponding to an additional dichlorobenzyaldehyde group (FIG. 5D). It was therefore reasoned that a possible addition of dichlorobenzyaldehyde group at the piperidine or pyrazole ring would match the molecular weight and synthesised both possible compounds, AP-5-144 and JMS-175-2 (FIG. 1C). The synthesis of the two isomers was confirmed using mass spectrometry fragmentation analyses (FIG. 5E) and the inhibitory effect of the isomers was tested against BRISC. Compound JMS-175-2 matched the compound in well H₂0, inhibiting BRISC with an IC50 of 1.5 μM (FIG. 1D). The other possible isomer, AP-5-144, showed no inhibition of BRISC DUB activity. These data confirm the chemical structure of the first BRISC inhibitor, JMS-175-2, identified due to a serendipitous addition of two dichlorobenzaldehyde groups during synthesis of compounds for the high throughput screen.

The specificity of JMS-175-2 for the BRISC DUB beyond USP2 and trypsin was also determined. JMS-175-2 exhibited high specificity for BRISC over other zinc-dependent DUBs in the JAMM/MPN family (FIG. 1E). JMS-175-2 showed no inhibition of AMSH-LP which, like BRCC36, specifically cleaves K63-linked polyubiquitin chains^{26,27}. Remarkably, JMS-175-2 specifically inhibits BRISC over the nuclear ARISC and ARISC-RAP80 complexes, which share three of the four BRISC subunits (FIG. 1E). These data confirm JMS-175-2 is a highly specific BRISC inhibitor and indicate the specificity is conferred from the Abraxas2 subunit which is replaced by Abraxas1 in the ARISC complex.

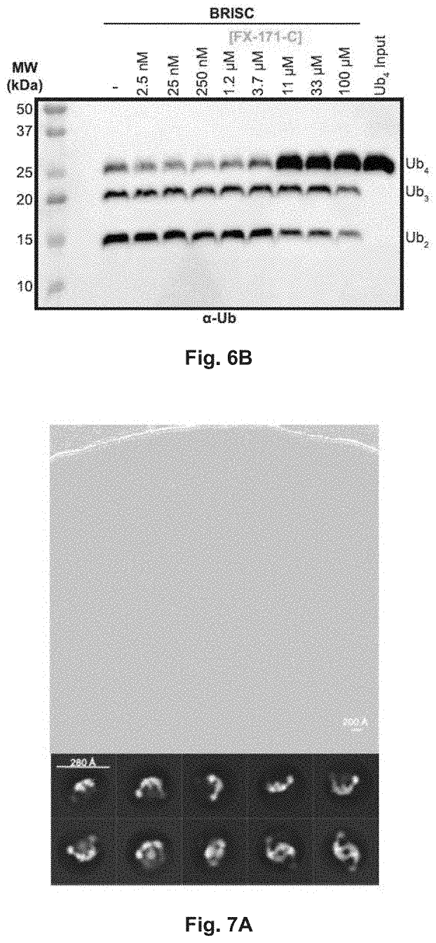

The JMS-175-2 compound was further optimised in a medicinal chemistry programme and SAR screening where a further 150 analogues of JMS-175-2 were synthesised. The potency of these analogues was characterised in the IQF assay with a fluorescent di-ubiquitin substrate. Inhibitor FX-171-C (the chemical structure of which is shown in FIG. 6A) was found to have slightly improved IC₅₀ compared to JMS-175-2 (FIG. 1F). We confirmed the ability of these inhibitors to block BRISC-mediated tetraubiquitin chain cleavage by Western blotting (FIG. 6B). Dose-dependent inhibition of tetraubiquitin chain cleavage for FX-171-C was observed, consistent with the di-ubiquitin fluorescence assay (FIG. 6B).

### Example 3—BRISC Inhibitors Induce Formation of a BRISC Dimer Complex

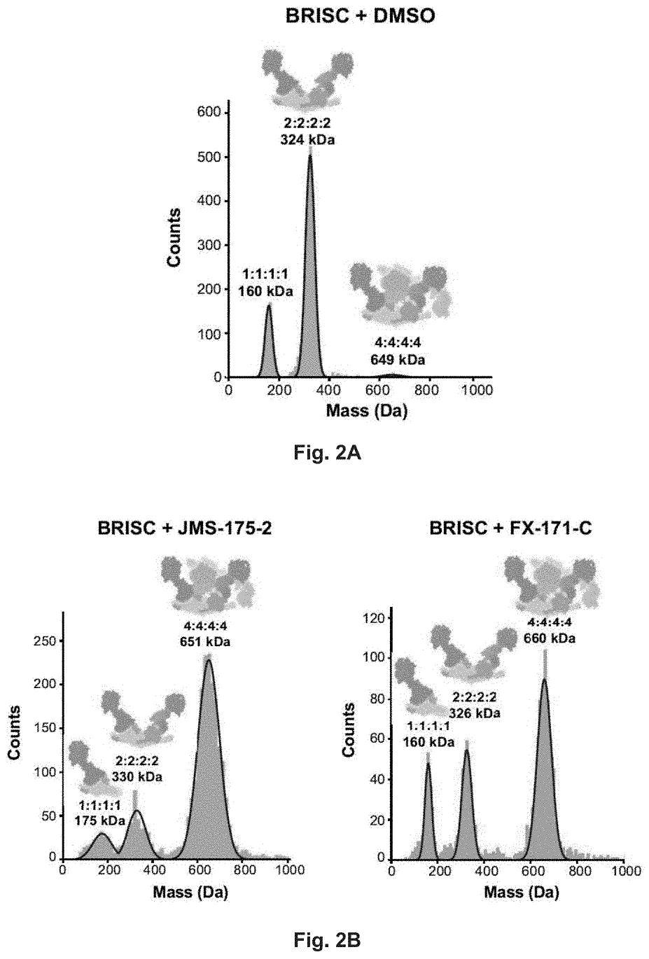

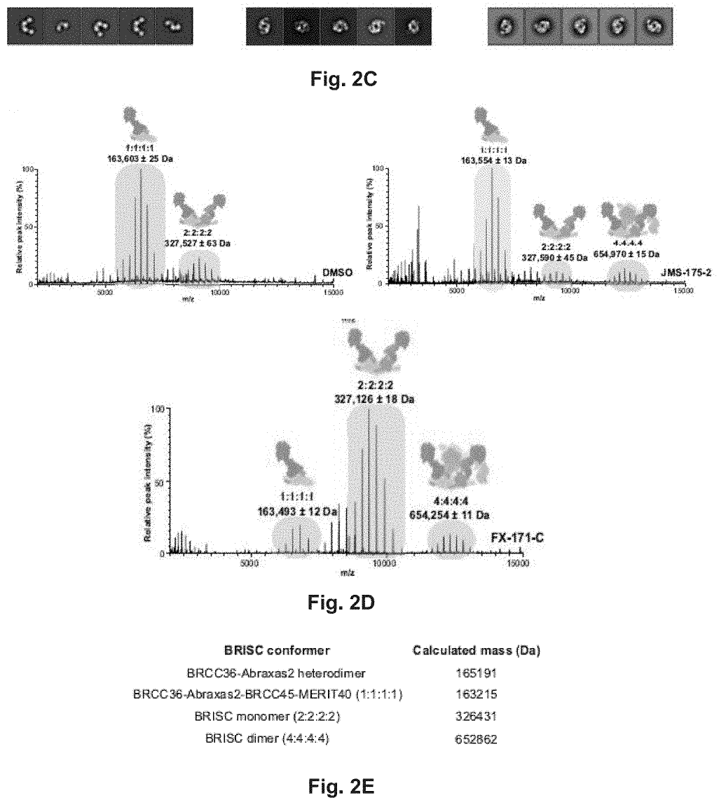

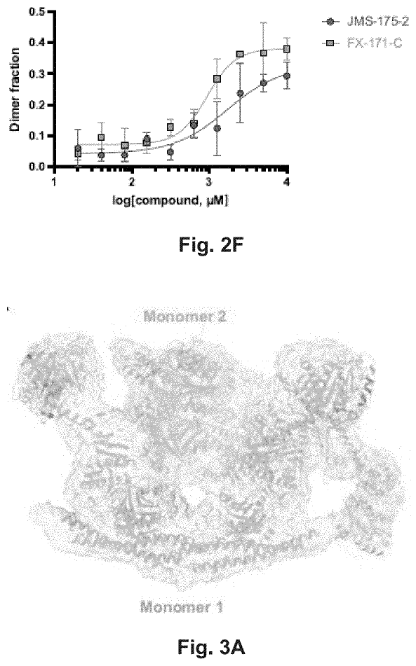

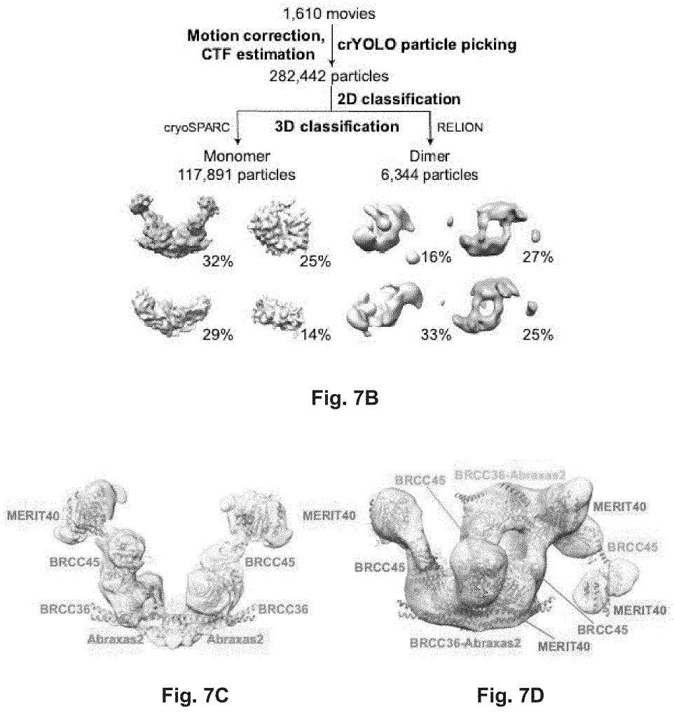

To understand the molecular basis of BRISC inhibition by the BLUE inhibitor series, and to determine the small molecule binding site, the complex by cryo-electron microscopy was characterised. During sample optimisation, the quality of the samples was we assessed by mass photometry, a single molecule mass measurement method. In mass photometry measurements of purified BRISC complex, it was surprising to observe three populations of the BRISC enzyme. Firstly, the major population corresponds to monomeric BRISC with four subunits at a 2:2:2:2 ratio (FIG. 2A), consistent with previous studies^{13,28,29}. In addition, a population at ˜160 kDa which corresponds to a 1:1:1:1 complex, or the BRCC36-Abraxas2 super dimer and minimally active complex was observed 12. A third population, consisting of ˜2% of the particles, had an estimated molecular weight of 649 kDa. This corresponds to the mass of two BRISC monomers (a 4:4:4:4 complex). This is consistent with cryoEM data, where BRISC “dimer” complexes was observed in 2D classes for ˜5% of the particles (FIGS. 7A, 7B). The majority of particles corresponded to a monomeric complex (FIG. 7C), but a low resolution cryoEM reconstruction of these particles indeed indicated density for two BRISC monomers (FIG. 7D). BRISC dimers have previously been reported where GraFix has been used during grid preparation¹⁵, but a more open, asymmetric dimer formation as observed in the present case.

When BRISC was mixed with the lead compound, JMS-175-2, and related inhibitor FX-171-C, a shift in mass to the 4:4:4:4 complex was observed, which suggested the inhibitor promoted BRISC dimer formation (FIG. 2B). Negative-stain electron microscopy was used to further probe the oligomeric state on addition of inhibitor and observed 2D class averages which look like two U-shaped BRISC assemblies interacting in the presence of JMS-157-2 and FX-171-C (FIG. 2C, middle and right panels). To confirm the observed inhibitor-induced mass corresponds to a dimeric full-length BRISC complex native mass spectrometry measurements were performed with and without inhibitors (FIG. 2D). BRISC dimers with a MW consistent with a 4:4:4:4 stoichiometry were observed for the JMS-175-2 and FX-171-C compounds. A dose-dependent increase in dimer formation by mass photometry for JMS-175-2 and FX-171-C was also observed (FIG. 2E). These data revealed a remarkable mode of action for BRISC small molecule inhibitors. It suggested JMS-175-2 and FX-171-C induce an autoinhibited BRISC dimer complex, whereby the adjacent BRISC monomer blocks the enzymes' active site, precludes binding of polyubiquitin substrates, and renders two catalytically inactive BRISC molecules.

### Example 4—BRISC Inhibitors Act as Molecular Glues

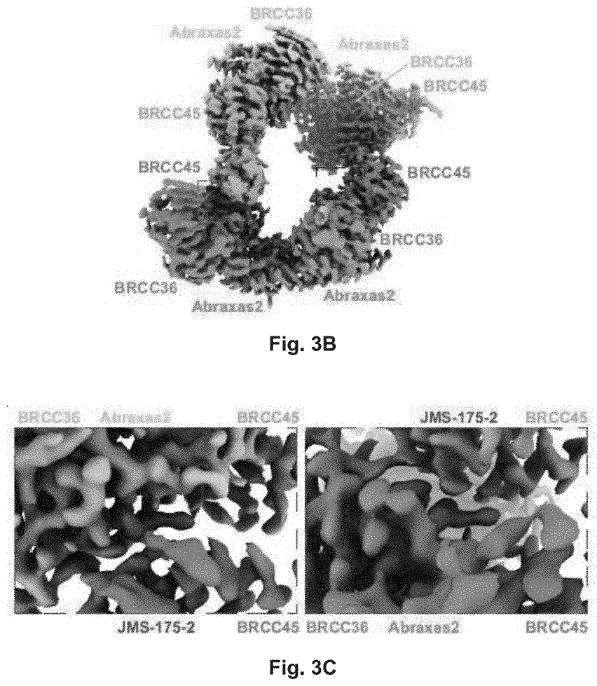

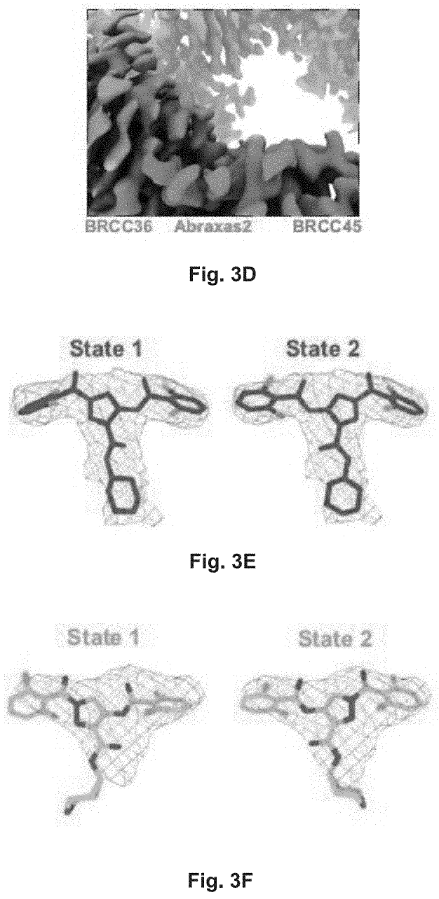

To determine the precise mechanism by which a small molecule can induce formation of a 650 kDa multimeric DUB complex, two structures of a BRISC-inhibitor complex, with inhibitors JMS-175-2 and FX-171-C, were solved. After 3D refinement and postprocessing, applying both C1 and C2 symmetry, maps at 3.5 Å (JMS-175-2) and 4.1 Å (FX-171-C) were achieved (FIG. 3, FIG. 8, FIG. 9). The BRISC-inhibitor structure is a dimer, with density for all 16-subunits of a dimeric complex, where the BRCC45-MERIT40 “arms” of one BRISC monomer hook around the BRCC45-MERIT40 arm of the neighbouring BRISC molecule, bridging the BRCC36-Abraxas2 superdimer (FIG. 3A). The highest resolution (3.26 Å-3.5 Å) in the core of the structure—consisting of the BRCC36-Abraxas2 superdimer and the BRCC45 subunits which form the dimer interface was observed. The resolution was lower (7-12 Å) for the extreme C-termini of BRCC45 and MERIT40 (arm regions) owing to the flexible nature of these regions and consistent with previous BRISC-SHMT2 cryo-EM structures¹³. Previous structures of the BRISC-SHMT2 complex^{13,15} were unambiguously fit into the BRISC dimer density. Due to the lower resolution of the data beyond the second ubiquitin E2 variant (UEV) domain of BRCC45, the structure a homology model of BRCC45 was modelled instead. The MERIT40 domain based on a BRISC-SHMT2 structure was modelled but with an approximate resolution of 8-12 Å in this region, the exact orientation of the von Willebrand factor type A (vWFA) domain of MERIT40 could not be determined.

The structures revealed the binding interface between two BRISC molecules, which is formed by BRCC36 and Abraxas2 from one BRISC monomer and BRCC45 from a second BRISC monomer. The same binding interface is formed on the opposite site of the dimer structure. additional density in these two regions was identified which cannot be attributed to either BRISC monomer (FIG. 3C). Importantly, the equivalent BRCC36-Abraxas2 surface that is not in contact with BRCC45 from an opposing BRISC monomer did not contain extra cryo-EM density (FIG. 3D). The extra density was present in the same location in both the JMS-175-2 and FX-171-C maps, indicating an equal mode of binding for these two inhibitors, and the stoichiometry of the BRISC-inhibitor complex 4:4:4:4:2 (i.e. one inhibitor molecule for each BRISC octamer). In each map, the observed density is in agreement with the JMS-175-2 and FX-171-C chemical structures respectively (FIGS. 3E, 3F). Due to the symmetry of the dichlorobenzene groups, the compounds were modelled as either binding in State 1 or State 2, as at such resolution it is not possible to unambiguously determine the precise orientation of the dichlorobenzene rings in the electron density. Focused refinement was applied to further improve the density for the inhibitor and surrounding residues. Applying a mask on the highest resolution half of the JMS-157-2 C1 map (FIG. 8E), improved the resolution to 3.3 Å, and at 3.2 Å in the very core of the enzyme (FIG. 8F).

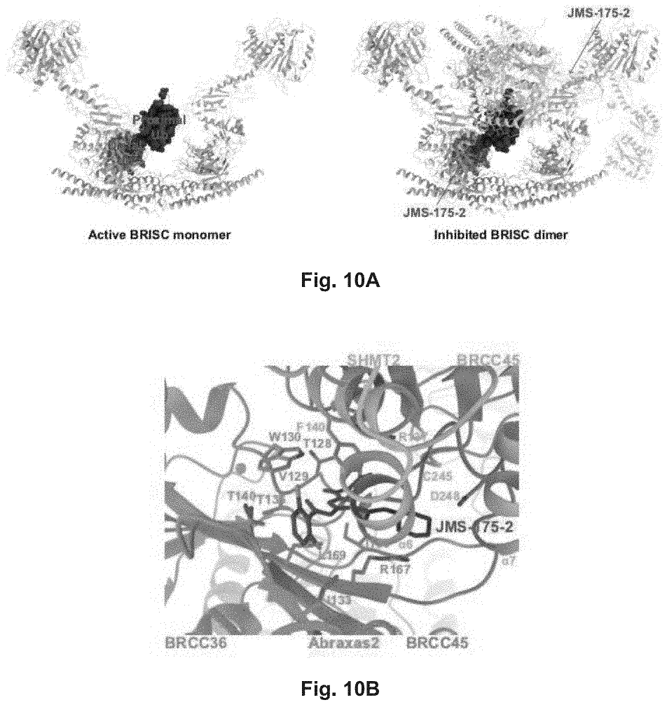

The mode of action of these inhibitors is evident from the structural analyses. BLUE compounds act as molecular glues to bridge together three proteins, BRCC36, Abraxas2, and BRCC45. This promoted formation of a dimeric complex, thereby blocking the BRCC36 active site and ubiquitin chain binding (FIG. 10A).

### Example 5—BRISC-Inhibitor Interactions

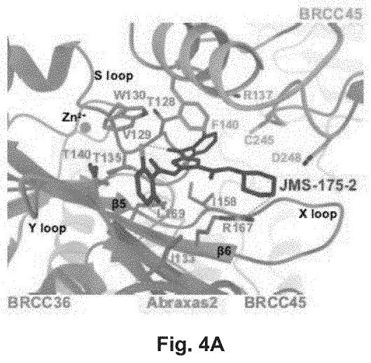

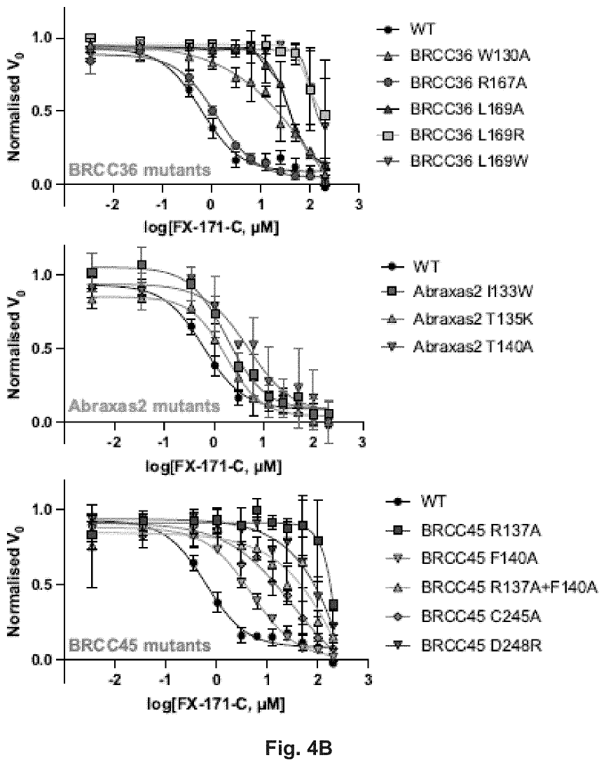

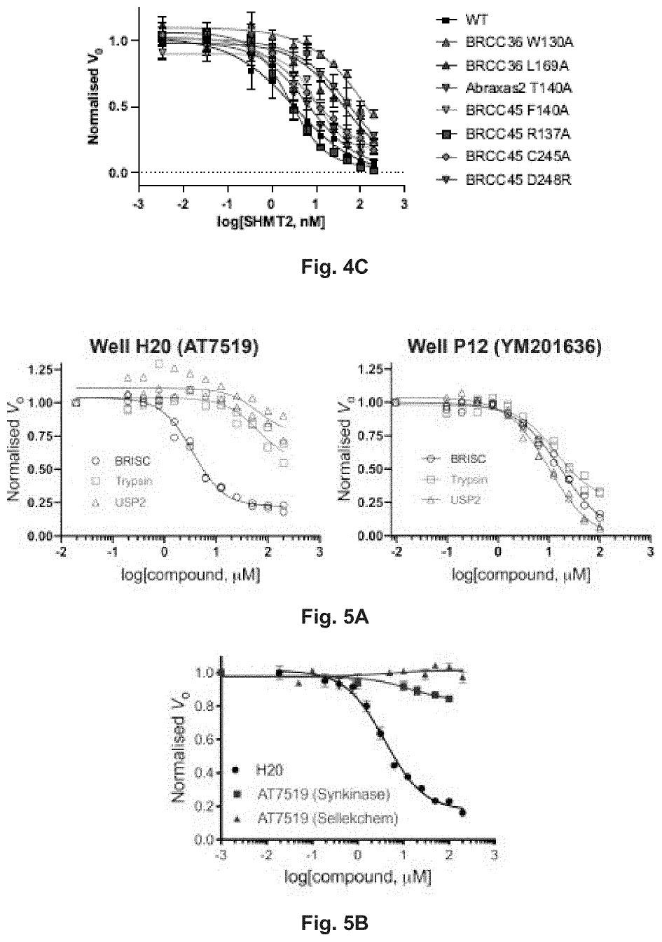

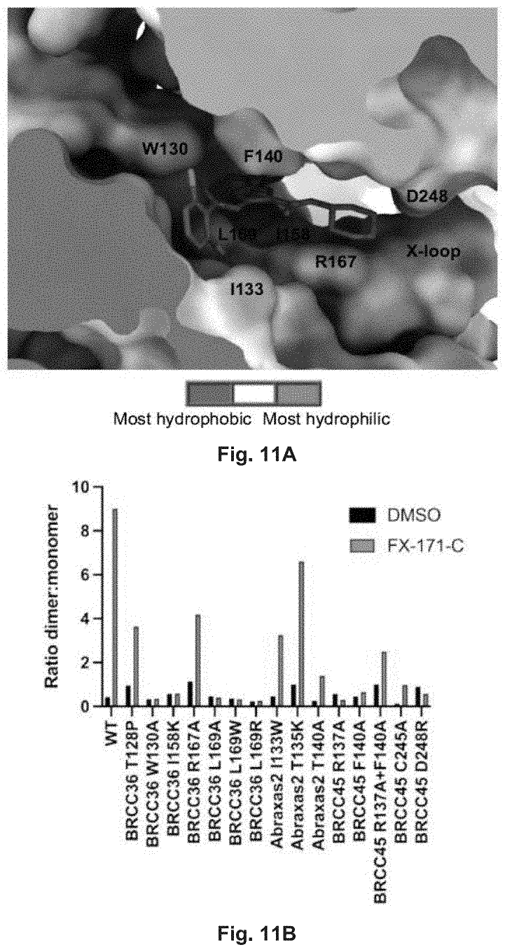

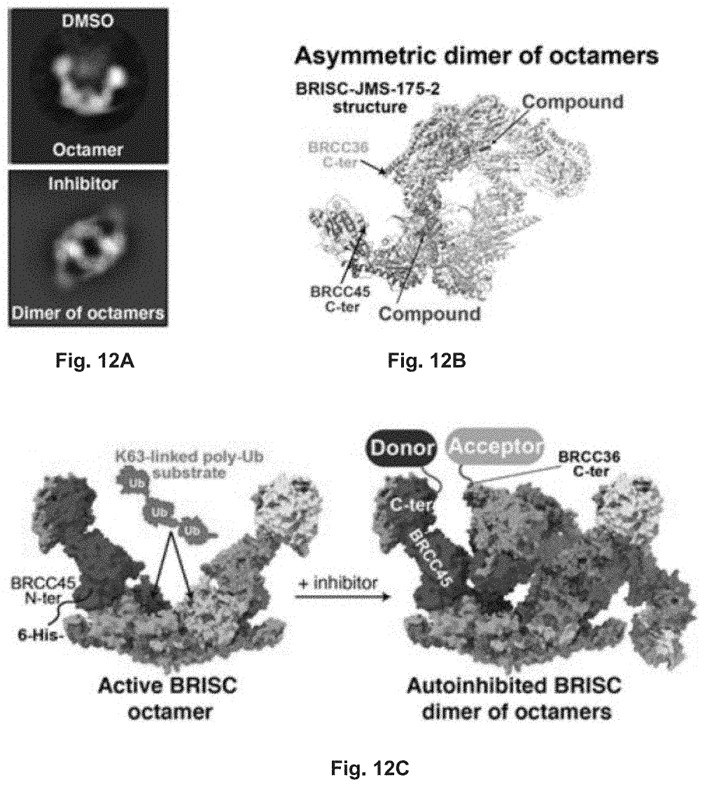

The high resolution of our cryo-EM structures at the dimer interface enabled identification of the residues from each BRISC monomer which contributed to inhibitor binding and molecular gluing. BLUE compounds sit at the interface of BRCC36 and Abraxas2 from one monomer, generating a binding surface for the BRCC45 subunit from an adjacent monomer. This triad of proteins forms an inhibitor binding cavity, with a buried surface area of 31.4 Å^{2 30}, in close proximity to the Zn²⁺ binding site of BRCC36 (FIG. 4A). The molecular glue forms a binding pocket between three flexible loop regions from BRCC36, Abraxas2 and two α-helices from BRCC45. The two dichlorobenzene moieties of JMS-175-2 and FX-171-C sit in a hydrophobic groove formed by BRCC36 S-loop residue, Trp130, and X-loop residues Ile158 and Leu169. The Abraxas2 Y-loop residue Ile133 and BRCC45 Phe140 also contribute to this hydrophobic groove (FIG. 11A). The X-loop (residues 155-169) between BRCC36 β-sheets, β-5 and β-6, forms a hydrophilic region into which the JMS-175-2 pyridine ring extends. Two hydrogen bonds are formed between JMS-175-2 and BRCC36. The amide backbone of Val129 (S-loop) forms a hydrogen bond with the carbonyl moiety of JMS-175-2 (modelled in state 2). Arg167 forms a second hydrogen bond with the amide of the pyridine ring³¹ (FIG. 4A).

Comparison of the AMSH-LP-diUb structure to the BRISC model revealed additional rationale for inhibition by BLUE compounds. The distal ubiquitin contacts the S-loop (FIG. 10A). This region becomes protected on the addition of FX-171-C in the HDX data, indicating that it becomes less solvent exposed and undergoes structural rearrangement upon BLUE compound binding. The inhibitor possibly blocks substrate binding in a two-fold manner. Dimer formation sterically occludes poly-ubiquitin processing, and small molecule binding to the S-loop causes local structural rearrangement to preclude substrate binding. Interestingly, comparison of the BRISC-inhibitor structure with the BRISC-SHMT2 structure also reveals that the binding site for the protein-based inhibitor, SHMT2, and the small molecule inhibitors overlap (FIG. 7B). The BRISC-SHMT2 interaction is required for targeting BRISC to trigger IFNAR1 deubiquitylation and stabilisation¹³. The overlap of the SHMT2 and BLUE binding sites suggest BLUE inhibition of BRISC would also disrupt BRISC targeting to substrates via SHMT2.

### Example 6—BRISC-Inhibitor Interacting Residues are Important for Inhibitor Sensitivity In Vitro

The cryo-EM structures of BRISC bound to molecular glues allowed picking of selective mutations to probe the BRISC-compound interaction site and to assess the contribution of each interacting residue for inhibition and gluing activity. Residues from BRCC36, Abraxas2 and BRCC45 were mutated and 15 mutant BRISC complexes were purified from insect cells (FIG. 11A). Due to the close proximity of some residues to the BRCC36 active site, BRISC DUB activity was assessed against a fluorogenic diUb substrate. Two mutants were inactive (BRCC36 Thr128Pro (S-loop mutant), Ile158Lys) (FIG. 11B). For the remaining active 13 mutants (FIGS. 11C, 11D) inhibitor IC50s were calculated against BRISC DUB activity. BRCC36 Trp130Ala and Leu169Ala mutants were severely affected for their ability to be inhibited by FX-171-C, whilst BRCC36 Arg167Ala remained inhibitor sensitive (FIG. 4B). Abraxas2 mutant Thr140Ala was moderately affected, exhibiting an IC50 10-fold higher than BRISC WT, and Abraxas2 Ile133Trp and Thr135Lys had no effect on inhibitor sensitivity (FIG. 4B). Four BRCC45 residues which contribute to glue activity were mutated and the ability to recognise the inhibitor was reduced (FIG. 4B). These results were corroborated by mass photometry measurements of the inhibitor-induced BRISC dimers which correlated with the IC50 measurements. A reduction in the inhibitor-induced dimerisation effect for the Ile158Lys mutant was also observed, but the Thr128Pro mutant still formed a dimer on the addition of FX-171-C, demonstrating that mass photometry is a suitable alternative assay to detect inhibitor sensitivity of inactive BRISC forms. Collectively, these data verify the BLUE compound binding site in vitro. Mutations in the BRCC36 S-loop and X-loop reduced compound sensitivity and dimer formation. Abraxas2 Y-loop mutants showed reduction in FX-171-C sensitivity but not complete loss of inhibition. Importantly, the insensitivity of the BRCC45 mutants confirmed the molecular gluing mode of inhibition. Interestingly, Arg167Ala shows comparable sensitivity to WT (FIG. 4C), which suggested the hydrogen bond formed between Arg167 and JMS-175-2 is not essential for inhibitor binding.

Due to the overlap of the SHMT2 binding site and small-molecule binding site, some of the mutated residues were observed to contribute to both interaction interfaces (FIG. 11B). As SHMT2 is a potent inhibitor of BRISC DUB activity¹³, whether BRISC mutants were still inhibited by SHMT2 was assessed (FIG. 4C). BRCC36 Trp130Ala and Leu169Ala and Abraxas2 Thr140Ala showed reduced inhibition in the presence of SHMT2, indicating that these mutations disrupt SHMT2 binding to BRISC. The BRCC45 mutants were inhibited with a similar IC50 to BRISC WT. In the previous BRISC-SHMT2 structure¹³ these residues were observed far from the BRISC-SHMT2 binding interface and were only brought in close proximity on the addition of BLUE compound.

### Example 9—Fluorescent Resonance Energy Transfer (FRET) and Bioluminescent Resonance Energy Transfer (BRET) Reporter Assays

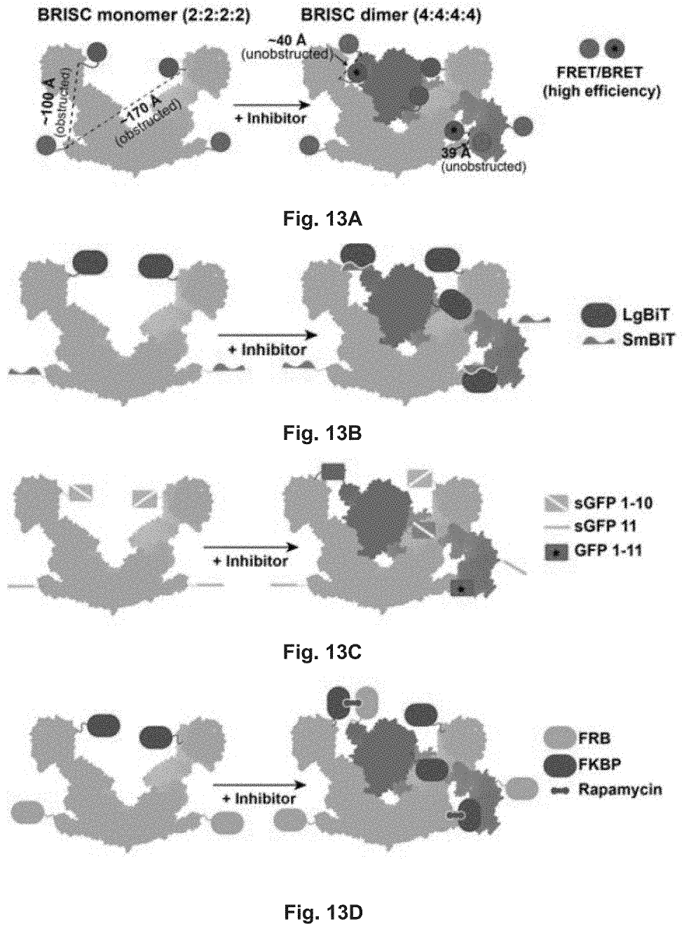

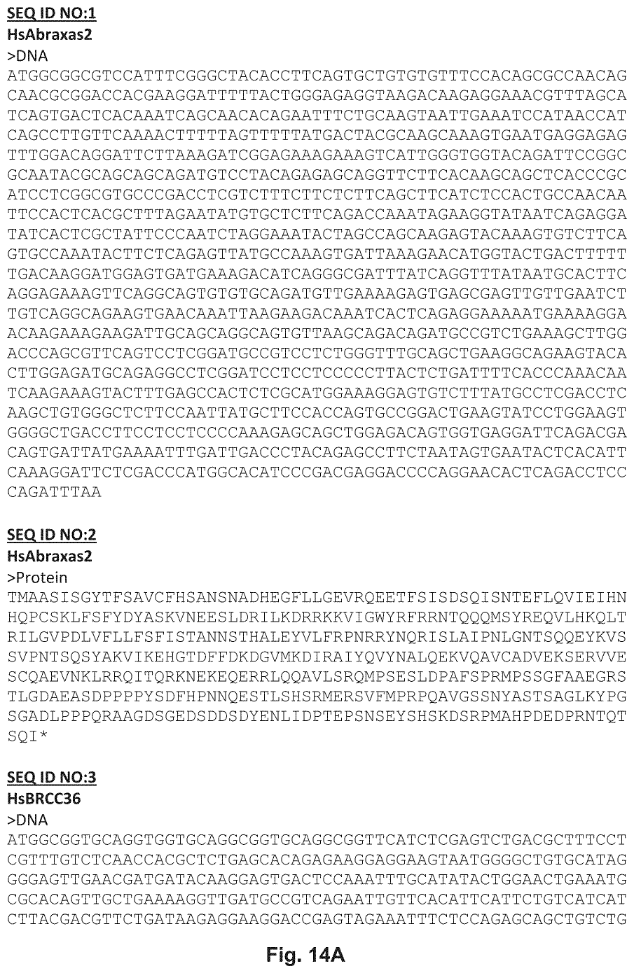

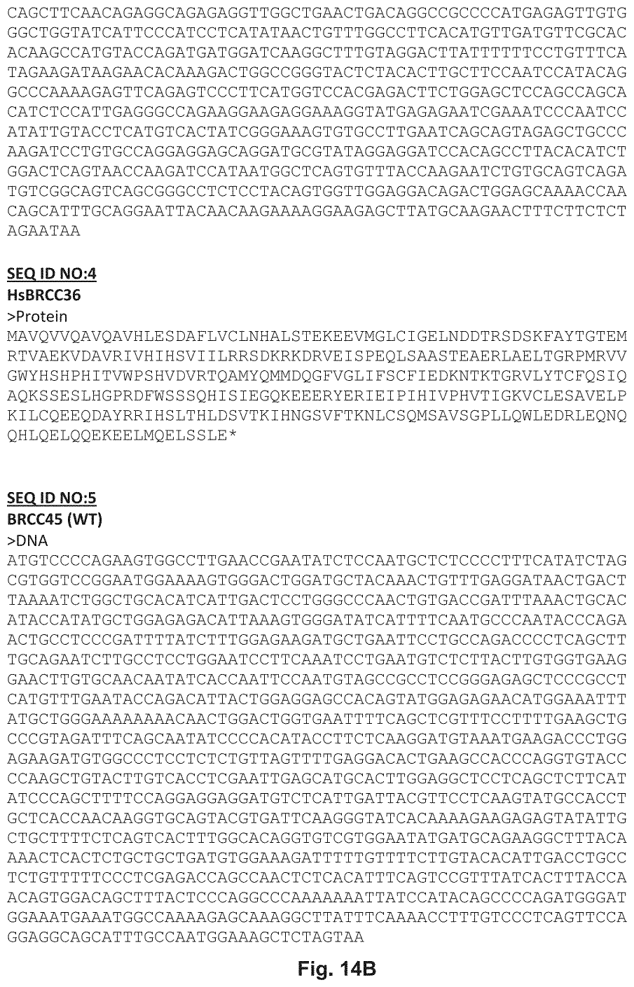

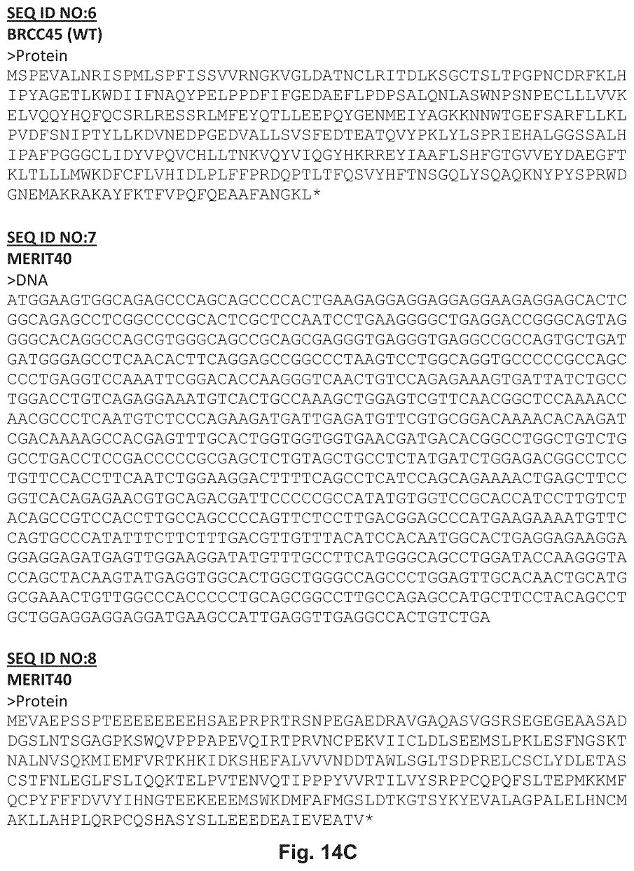

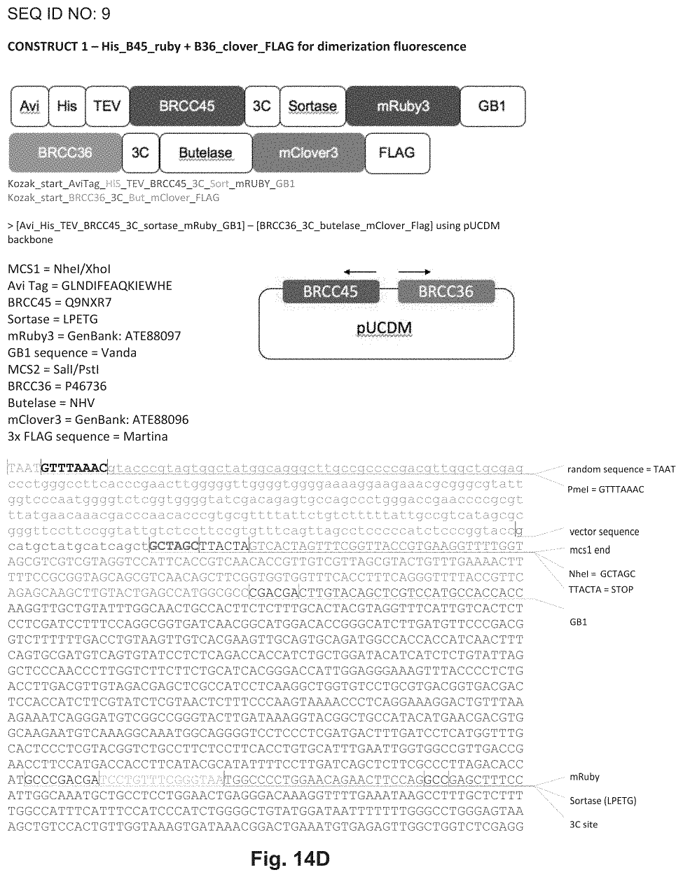

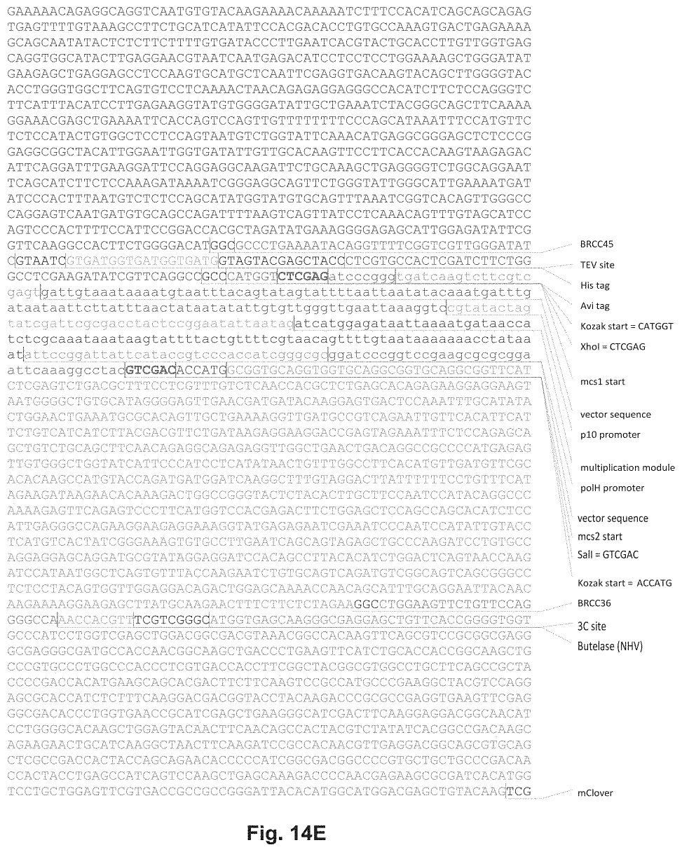

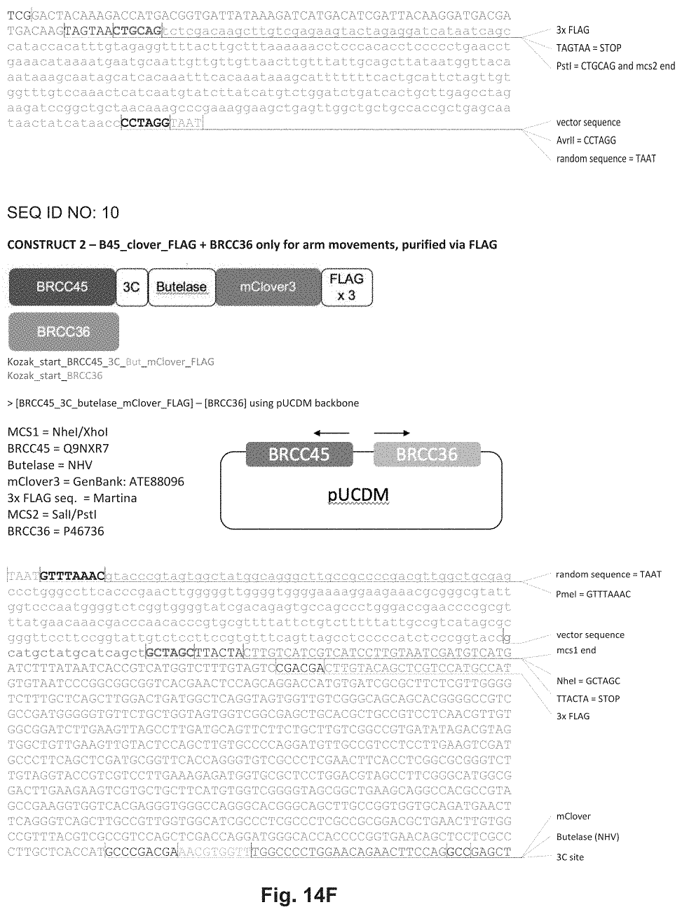

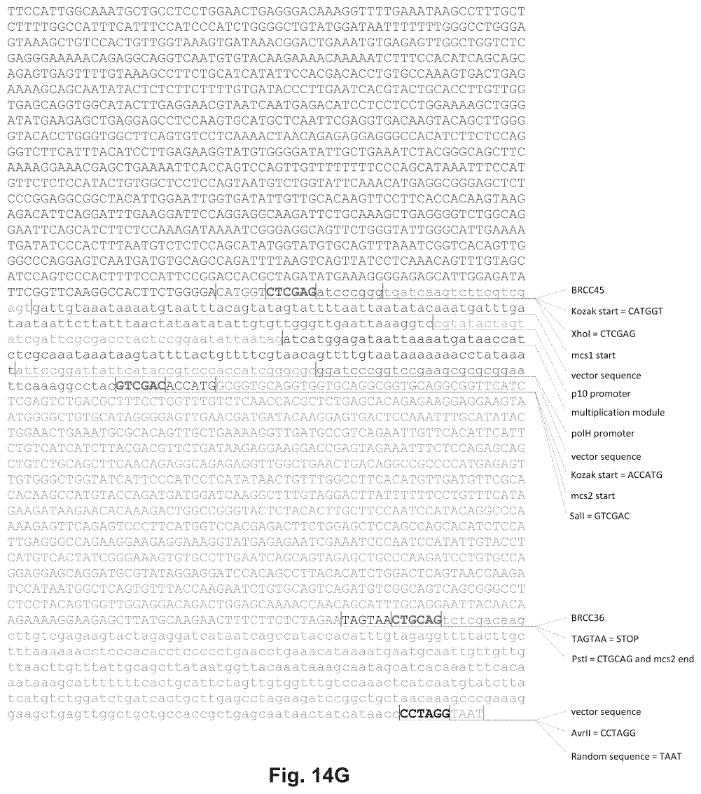

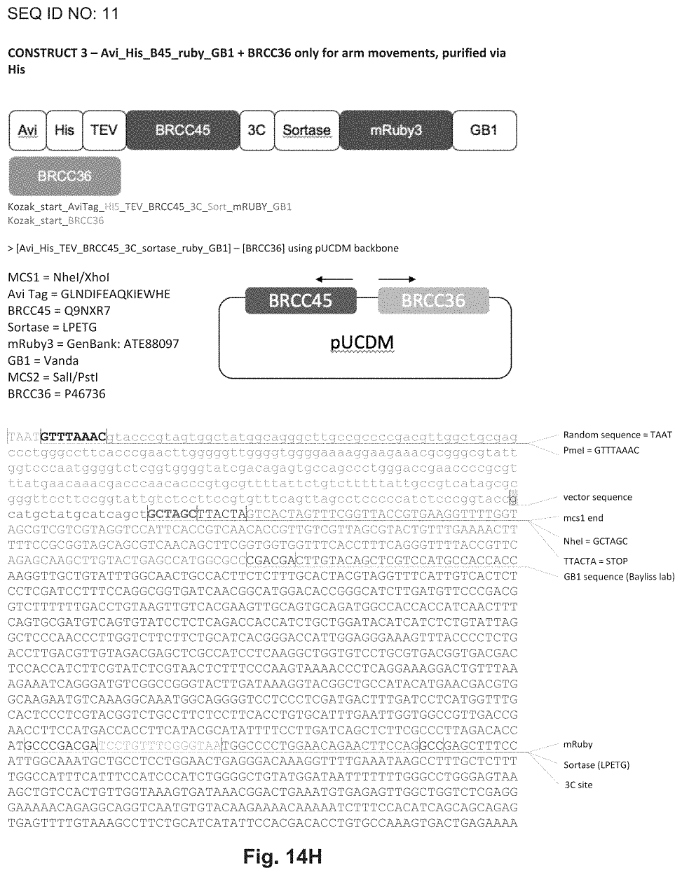

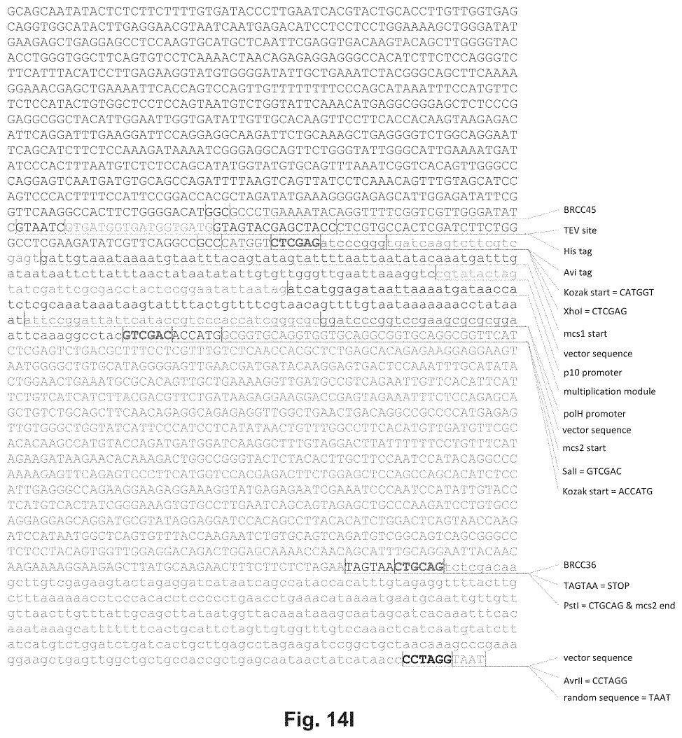

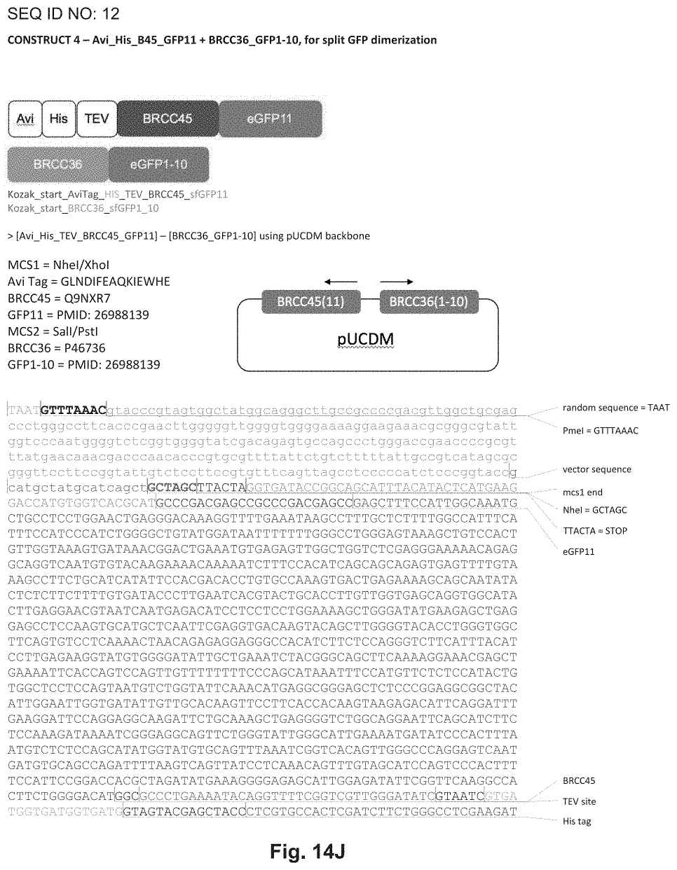

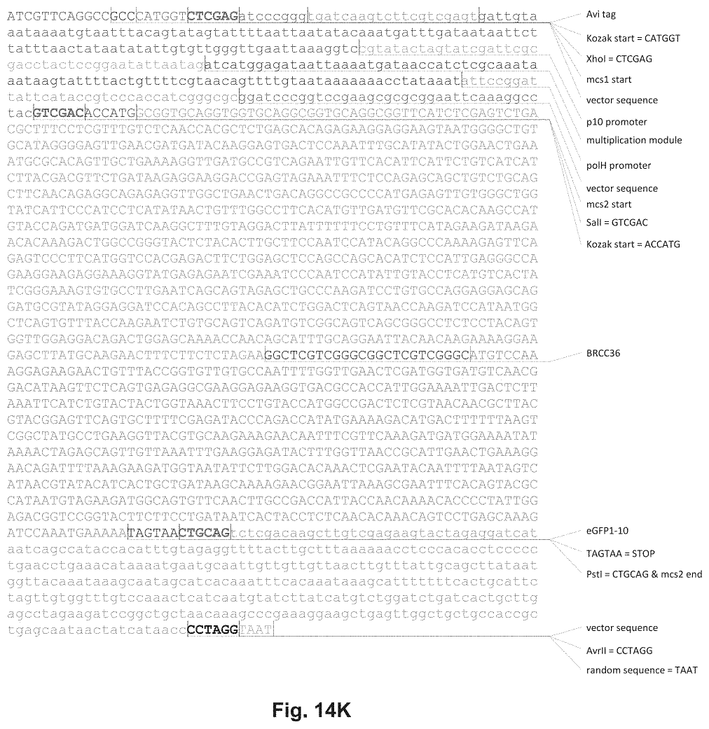

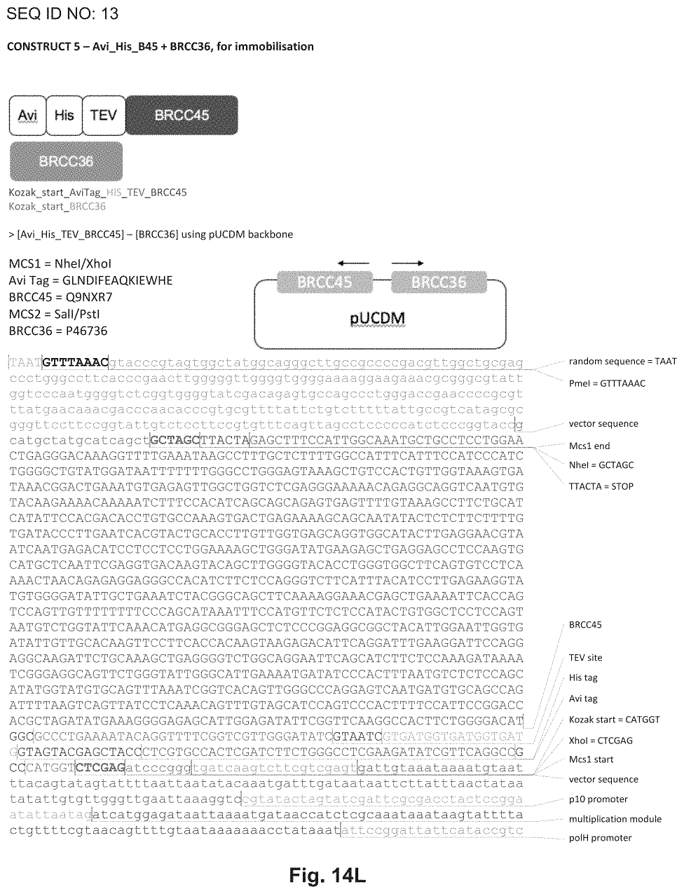

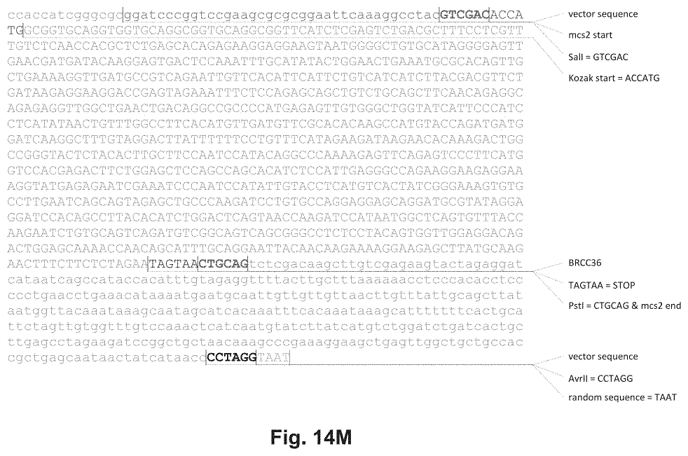

Addition of fluorescent and/or luminescent reporters were generated by tagging the BRCC45 and BRCC36 C-termini with fluorescent and/or luminescent tags using standard gene synthesis and cloning procedures. GS linkers (2-6 amino acids long) and/or epitope tags such as Flag were used to fuse BRCC36 and BRCC45 with reporter tags (FIGS. 12 and 13). FRET pairs included mColver and mRuby and luminescent tags included large and small fragments of nanoluciferase (Dixon et al., 2015). This assay was used to screen for new molecular glues that bind BRISC and cause dimerization in vitro and in cells (FIG. 13). A split GFP system was also used as a reporter for dimerization (FIG. 13C).

### Example 10—Generation of a Stable BRISC Dimer Via Specific Linkers and Tags

Stable BRISC dimers were generated using specific linkers and tags fused to specific BRISC subunits (FIGS. 13C and D). This resulted in more stable protein samples that were more amenable to crystallisation and/or cryoEM sample preparation to obtain co-structures with BRISC and BRISC inhibitors/molecular glues. A stable BRISC dimer was induced using a split GFP construct by fusing beta1-10 to the C-terminus of BRCC45 and beta11 to the C-terminus of BRCC36 using a Gly-Ser linker (FIG. 13C). A similar strategy was followed to generate a Rapamycin-inducible FRB-FKBP dimer (FIG. 13C).

### REFERENCES

1. Hershko, A. & Ciechanover, A. The ubiquitin system. Annu. Rev. Biochem 67, 425-79 (1998).
2. Hu, H. & Sun, S. C. Ubiquitin signaling in immune responses. Cell Research 26, 457-483 (2016).
3. Ross, C. A. & Pickart, C. M. The ubiquitin-proteasome pathway in Parkinson's disease and other neurodegenerative diseases. Trends in Cell Biology 14, 703-711 (2004).
4. Sacco, J. J., Coulson, J. M., Clague, M. J. & Urbé, S. Emerging roles of deubiquitinases in cancer-associated pathways. IUBMB Life 62, 140-157 (2010).
5. Cohen, P. & Tcherpakov, M. Will the ubiquitin system furnish as many drug targets as protein kinases? Cell 143, 686-693 (2010).
6. Harrigan, J. A., Jacq, X., Martin, N. M. & Jackson, S. P. Deubiquitylating enzymes and drug discovery: Emerging opportunities. Nat. Rev. Drug Discov. 17, 57-77 (2018).
7. Cooper, E. M. et al. K63-specific deubiquitination by two JAMM/MPN+ complexes: BRISC-associated Brcc36 and proteasomal Poh1. EMBO J. 28, 621-631 (2009).
8. Feng, L., Wang, J. & Chen, J. The Lys63-specific deubiquitinating enzyme BRCC36 is regulated by two scaffold proteins localizing in different subcellular compartments. J. Biol. Chem. 285, 30982-30988 (2010).
9. Kim, H., Chen, J. & Yu, X. Ubiquitin-binding protein RAP80 mediates BRCA1-dependent DNA damage response. Science (80-.). 316, 1202-1205 (2007).
10. Sobhian, B. et al. RAP80 targets BRCA1 to specific ubiquitin structures at DNA damage sites. Science (80-.). 316, 1198-1202 (2007).
11. Wang, B. et al. Abraxas and RAP80 form a BRCA1 protein complex required for the DNA damage response. Science (80-.). 316, 1194-1198 (2007).
12. Zeqiraj, E. et al. Higher-Order Assembly of BRCC36-KIAA0157 Is Required for DUB Activity and Biological Function. Mol. Cell 59, 970-983 (2015).
13. Walden, M. et al. Metabolic control of BRISC-SHMT2 assembly regulates immune signalling. Nature 570, 194-199 (2019).
14. Kyrieleis, 0. J. P. et al. Three-Dimensional Architecture of the Human BRCA1-A Histone Deubiquitinase Core Complex. CellReports 17, 3099-3106 (2016).
15. Rabl, J. et al. Structural Basis of BRCC36 Function in DNA Repair and Immune Regulation. Mol. Cell 75, 483-497 (2019).
16. Zheng, H. et al. A BRISC-SHMT Complex Deubiquitinates IFNAR1 and Regulates Interferon Responses. Cell Rep. 5, 180-193 (2013).
17. Pascual, V., Farkas, L. & Banchereau, J. Systemic lupus erythematosus: all roads lead to type I interferons. Current Opinion in Immunology 18, 676-682 (2006).
18. Conigliaro, P. et al. The type I IFN system in rheumatoid arthritis. in Autoimmunity 43, 220-225 (Autoimmunity, 2010).
19. Wu, M. & Assassi, S. The role of type 1 interferon in systemic sclerosis. Frontiers in Immunology 4, (2013).
20. Lauinger, L. et al. Thiolutin is a zinc chelator that inhibits the Rpn11 and other JAMM metalloproteases. Nat. Chem. Biol. 13, 709-714 (2017).
21. Li, J. et al. Capzimin is a potent and specific inhibitor of proteasome isopeptidase Rpn11. Nat. Chem. Biol. 13, 486-493 (2017).
22. Schlierf, A. et al. Targeted inhibition of the COP9 signalosome for treatment of cancer. Nat. Commun. 7, 1-10 (2016).
23. Lange, S. M., Armstrong, L. A. & Kulathu, Y. Deubiquitinases: From mechanisms to their inhibition by small molecules. Mol. Cell 82, 15-29 (2022).
24. Ernst, A. et al. A strategy for Modulation of Enzymes in the Ubiquitin System. 209, 590-596 (2013).
25. Guo, Y. et al. Structural and functional characterization of ubiquitin variant inhibitors for the JAMM-family deubiquitinases STAMBP and STAMBPL1. J. Biol. Chem. 297, 101107 (2021).
26. McCullough, J., Clague, M. J. & Urbé, S. AMSH is an endosome-associated ubiquitin isopeptidase. J. Cell Biol. 166, 487-492 (2004).
27. Sato, Y. et al. Structural basis for specific cleavage of Lys 63-linked polyubiquitin chains. Nature 455, 358-362 (2008).
28. Patterson-Fortin, J., Shao, G., Bretscher, H., Messick, T. E. & Greenberg, R. A. Differential regulation of JAMM domain deubiquitinating enzyme activity within the RAP80 complex. J. Biol. Chem. (2010). doi:10.1074/jbc.M110.135319
29. Kyrieleis, O. J. P. et al. Three-Dimensional Architecture of the Human BRCA1-A Histone Deubiquitinase Core Complex. Cell Rep. (2016). doi:10.1016/j.celrep.2016.11.063
30. Krissinel, E. & Henrick, K. Inference of macromolecular assemblies from crystalline state. J. Mol. Biol. 372, 774-797 (2007).
31. Adasme, M. F. et al. PLIP 2021: expanding the scope of the protein-ligand interaction profiler to DNA and RNA. Nucleic Acids Res. 49, W530-W534 (2021).
32. Fitzgerald, D. J. et al. Protein complex expression by using multigene baculoviral vectors. Nat. Methods 3, 1021-1032 (2006).
33. Sonn-Segev, A. et al. Quantifying the heterogeneity of macromolecular machines by mass photometry. Nat. Commun. 11, (2020).
34. Zivanov, J. et al. New tools for automated high-resolution cryo-EM structure determination in RELION-3. Elife 7, (2018).
35. Zivanov, J., Nakane, T. & Scheres, S. H. W. Estimation of high-order aberrations and anisotropic magnification from cryo-EM data sets in RELION-3.1. IUCrJ 7, 253-267 (2020).
36. Thompson, R. F., Iadanza, M. G., Hesketh, E. L., Rawson, S. & Ranson, N. A. Collection, pre-processing and on-the-fly analysis of data for high-resolution, single-particle cryo-electron microscopy. Nat. Protoc. 14, 100-118 (2019).
37. Zheng, S. Q. et al. MotionCor2: anisotropic correction of beam-induced motion for improved cryo-electron microscopy. Nat. Methods 14, 331-332 (2017).
38. Zhang, K. Gctf: Real-time CTF determination and correction. J. Struct. Biol. 193, 1-12 (2016).
39. Scarff, C. A., Fuller, M. J. G., Thompson, R. F. & Iadaza, M. G. Variations on negative stain electron microscopy methods: Tools for tackling challenging systems. J. Vis. Exp. 2018, (2018).
40. Wagner, T. et al. SPHIRE-crYOLO is a fast and accurate fully automated particle picker for cryo-EM. Commun. Biol. 2019 21 2, 1-13 (2019).
41. Pettersen, E. F. et al. UCSF Chimera—a visualization system for exploratory research and analysis. J. Comput. Chem. 25, 1605-1612 (2004).
42. Pettersen, E. F. et al. UCSF ChimeraX: Structure visualization for researchers, educators, and developers. Protein Sci. 30, 70-82 (2021).
43. Emsley, P., Lohkamp, B., Scott, W. G. & Cowtan, K. Features and development of Coot. Acta Crystallogr. Sect. D Biol. Crystallogr. 66, 486-501 (2010).
44. Adams, P. D. et al. PHENIX: A comprehensive Python-based system for macromolecular structure solution. Acta Crystallogr. Sect. D Biol. Crystallogr. 66, 213-221 (2010).
45. Schüttelkopf, A. W. & Van Aalten, D. M. F. PRODRG: A tool for high-throughput crystallography of protein-ligand complexes. Acta Crystallogr. Sect. D Biol. Crystallogr. 60, 1355-1363 (2004).
46. Lau, A. M. C., Ahdash, Z., Martens, C. & Politis, A. Deuteros: Software for rapid analysis and visualization of data from differential hydrogen deuterium exchange-mass spectrometry. Bioinformatics 35, 3171-3173 (2019).
47. Lau, A. M., Claesen, J., Hansen, K. & Politis, A. Deuteros 2.0: Peptide-level significance testing of data from hydrogen deuterium exchange mass spectrometry. Bioinformatics 37, 270-272 (2021).
48. Masson, G. R. et al. Recommendations for performing, interpreting and reporting hydrogen deuterium exchange mass spectrometry (HDX-MS) experiments. Nat. Methods 16, 595-602 (2019).

### OTHER EMBODIMENTS

In some embodiments, the present disclosure provides:
1. A method of identifying a selective BRISC inhibitor comprising:
   (i) contacting a population of BRISC monomers with a test compound; and
   (ii) measuring the level of BRISC dimers before and after step (i),
   wherein an increase in the level of BRISC dimers after contacting the population of BRISC monomers with the test compound identifies the test compound as a selective BRISC inhibitor.
2. The method of paragraph 1, wherein the BRISC monomers comprise an octameric complex comprising the subunits, Abraxas2, BRCC36, BRCC45 and MERIT40 at a 2:2:2:2 ratio.
3. The method of paragraph 1 or 2, wherein the BRISC dimers comprise a 16-meric complex comprising the subunits, Abraxas2, BRCC36, BRCC45 and MERIT40 at a 4:4:4:4 ratio.
4. The method of any one of the preceding paragraphs, wherein the Abraxas2 subunit is encoded by a nucleic acid sequence that is at least 85%, 90%, 95%, 96%, 97%, 98%, or 99% identical to the sequence of SEQ ID NO: 1.
5. The method of any one of the preceding paragraphs, wherein the BRCC36 subunit is encoded by a nucleic acid sequence that is at least 85%, 90%, 95%, 96%, 97%, 98%, or 99% identical to the sequence of SEQ ID NO: 3.
7. The method of any one of the preceding paragraphs, wherein the BRCC45 subunit is encoded by a nucleic acid sequence that is at least 85%, 90%, 95%, 96%, 97%, 98%, or 99% identical to the sequence of SEQ ID NO:5.
8. The method of any one of the preceding paragraphs, wherein the MERIT40 subunit is encoded by a nucleic acid sequence that is at least 85%, 90%, 95%, 96%, 97%, 98%, or 99% identical to the sequence of SEQ ID NO:7.
9. The method of any one of the preceding paragraphs, wherein one or more subunits of the one or more BRISC monomers is/are labelled with a first detection label from a detection pair and wherein one or more subunits of the one or more BRISC monomers is labelled with a second detection label from the detection pair.
10. The method of paragraph 9, wherein the detection pair comprises a fluorescent detection pair and/or one or a luminescent detection pair.
11. The method of paragraph 9 or 10, wherein the first detection label comprises a donor and the second detection label comprises an acceptor.
12. The method of any one of paragraphs 9-11, wherein the detection pair comprise a Fluorescent resonance energy transfer (FRET) pair or a Bioluminescence resonant energy transfer (BRET) pair.
13. The method of paragraph 12 wherein the FRET pair comprises mClover-mRuby, CyPet-YPet, EGFP-mCherry, Venus-mCherry, Venus-tdTomato, Venus-mPlum, EBFP2-mEGFP, ECFP-EYFP, Cerulean-Venus, MiCy-mKO or TFP1-mVenus, optionally mClover3-mRuby3.
14. The method of paragraph 12, wherein the BRET pair comprises Nanoluciferase-mCherry, Nanoluciferase-HaloTag, Nanoluciferase-Venus, Luciferase-GFP, Luciferase-YFP, Luciferase-Venus or LgiT-smBiT.
15. The method of any one of any one of paragraphs 9-14, wherein the donor is attached to the C-terminus of the BRCC45 subunit and/or the acceptor is attached to the C-terminus of the BRCC36 subunit, optionally wherein the donor is attached to position 383 of BRCC45 such as L383 and/or the acceptor is attached to position 316 of BRCC36 such as E316.
16. The method of any one of any one of paragraphs 9-14, wherein the acceptor is attached to the C-terminus of the BRCC45 subunit and/or the donor is attached to the C-terminus of the BRCC36 subunit, optionally wherein the acceptor is attached to position 383 of BRCC45 such as L383 and/or the donor is attached to position 316 of BRCC36 such as E316.
17. The method of paragraph 9, wherein the fluorescent detection pair comprises a split fluorescent molecule, optionally a split GFP molecule or a spyTag catcher pair.
18. The method of paragraph 17, wherein one half of the split fluorescent molecule is attached to the C-terminus of the BRCC45 subunit as the first detection label and the other half of the split fluorescent molecule is attached to the C-terminus of the BRCC36 subunit as the second detection label.
19. The method of paragraph 7, wherein the one or more subunits of BRISC is encoded by a nucleotide sequence comprising the sequence of SEQ ID NO: 9, SEQ ID NO: 10, SEQ ID NO: 11, SEQ ID NO: 12 or SEQ ID NO: 13.
20. The method of any one of the preceding paragraphs, wherein the formation of BRISC dimers can be detected by (i) mass spectrometry and/or (ii) measuring fluorescence signals and/or luminescence signals before and after addition of the compound, wherein for (ii) a relative increase in fluorescence signals and/or luminescence signals indicates an increase in the level of BRISC dimers or an increase in the fluorescence signal of the acceptor together with a decrease in the fluorescence signal of the donor indicates an increase in the level of BRISC dimers.
21. The method of any one of the preceding paragraphs, wherein an increase in the level of BRISC dimers of about 10% of BRISC after contacting the population of BRISC monomers with the test compound identifies the test compound as a selective BRISC inhibitor.
22. A BRISC dimer comprising:
   (i) a first detection label attached to a first BRISC monomer; and
   (ii) a second detection label attached to a second BRISC monomer, wherein the first detection label of (i) and the second detection label of (ii) are fused to each other.
23. The BRISC dimer of paragraph 22, wherein the first detection label and the second detection label are fused to each other by an intermediate molecule.
24. The BRISC dimer of paragraph 22 or 23, wherein first detection label is beta1-10 of GFP and the second detection label is beta 11 of GFP and the intermediate molecule is a Gly-Ser linker.
25. The BRISC dimer of any one of paragraphs 22-24, wherein the first detection label is FRB and the second detection label is FKBP and the intermediate molecule is rapamycin.
26. The BRISC dimer of any one of paragraphs 22-25, wherein each BRISC monomer comprises an octameric complex comprising the subunits, Abraxas2, BRCC36, BRCC45 and MERIT40 at a 2:2:2:2 ratio and the BRISC dimer comprises a 16-meric complex comprising the subunits, Abraxas2, BRCC36, BRCC45 and MERIT40 at a 4:4:4:4 ratio.
27. The BRISC dimer of any one of the preceding paragraphs, wherein the first detection label is attached to the C-terminus of BRCC45 of the first BRISC monomer and the second detection label is attached the C-terminus of BRCC36 of a second BRISC monomer.
28. Use of a BRISC dimer as defined by any one of paragraphs 22-27 to generate cryo-Electron Microscopy (cryo-EM), crystallography, nuclear magnetic resonance and/or X-ray crystallography structures for structure guided drug design.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of identifying a selective BRISC inhibitor comprising:
(i) contacting a population of BRISC monomers with a test compound; and
(ii) measuring the level of BRISC dimers before and after step (i),
wherein an increase in the level of BRISC dimers after contacting the population of BRISC monomers with the test compound identifies the test compound as a selective BRISC inhibitor.

2. The method of claim 1, wherein (i) the BRISC monomers comprise an octameric complex comprising the subunits, Abraxas2, BRCC36, BRCC45 and MERIT40 at a 2:2:2:2 ratio; or (ii) wherein the BRISC dimers comprise a 16-meric complex comprising the subunits, Abraxas2, BRCC36, BRCC45 and MERIT40 at a 4:4:4:4 ratio.

3. The method of claim 1, wherein
(i) the Abraxas2 subunit is encoded by a nucleic acid sequence that is at least 85%, 90%, 95%, 96%, 97%, 98%, or 99% identical to the sequence of SEQ ID NO: 1;
(ii) the BRCC36 subunit is encoded by a nucleic acid sequence that is at least 85%, 90%, 95%, 96%, 97%, 98%, or 99% identical to the sequence of SEQ ID NO: 3;
(iii) the BRCC45 subunit is encoded by a nucleic acid sequence that is at least 85%, 90%, 95%, 96%, 97%, 98%, or 99% identical to the sequence of SEQ ID NO:5; or
(iv) the MERIT40 subunit is encoded by a nucleic acid sequence that is at least 85%, 90%, 95%, 96%, 97%, 98%, or 99% identical to the sequence of SEQ ID NO:7.

4. The method of claim 1, wherein one or more subunits of the one or more BRISC monomers is/are labelled with a first detection label from a detection pair and wherein one or more subunits of the one or more BRISC monomers is labelled with a second detection label from the detection pair.

5. The method of claim 4, wherein the detection pair comprises a fluorescent detection pair and/or one or a luminescent detection pair.

6. The method of claim 4, wherein the first detection label comprises a donor and the second detection label comprises an acceptor.

7. The method of claim 4, wherein the detection pair (i) comprise a Fluorescent resonance energy transfer (FRET) pair, optionally wherein the FRET pair comprises mClover-mRuby, CyPet-YPet, EGFP-mCherry, Venus-mCherry, Venus-tdTomato, Venus-mPlum, EBFP2-mEGFP, ECFP-EYFP, Cerulean-Venus, MiCy-mKO, or TFP1-mVenus, optionally mClover3-mRuby3 or (ii) comprise a Bioluminescence resonant energy transfer (BRET) pair, optionally wherein the BRET pair comprises Nanoluciferase-mCherry, Nanoluciferase-HaloTag, Nanoluciferase-Venus, Luciferase-GFP, Luciferase-YFP, Luciferase-Venus or LgiT-smBiT.

8. The method of claim 6, wherein the donor is attached to the C-terminus of the BRCC45 subunit and/or the acceptor is attached to the C-terminus of the BRCC36 subunit, optionally wherein the donor is attached to position 383 of BRCC45 such as L383 and/or the acceptor is attached to position 316 of BRCC36 such as E316.

9. The method of claim 6, wherein the acceptor is attached to the C-terminus of the BRCC45 subunit and/or the donor is attached to the C-terminus of the BRCC36 subunit, optionally wherein the acceptor is attached to position 383 of BRCC45 such as L383 and/or the donor is attached to position 316 of BRCC36 such as E316.

10. The method of claim 5, wherein the fluorescent detection pair comprises a split fluorescent molecule, optionally a split GFP molecule or a spyTag catcher pair.

11. The method of claim 10, wherein one half of the split fluorescent molecule is attached to the C-terminus of the BRCC45 subunit as the first detection label and the other half of the split fluorescent molecule is attached to the C-terminus of the BRCC36 subunit as the second detection label.

12. The method of claim 3, wherein the one or more subunits of BRISC is encoded by a nucleotide sequence comprising the sequence of SEQ ID NO: 9, SEQ ID NO: 10, SEQ ID NO: 11, SEQ ID NO: 12 or SEQ ID NO: 13.

13. The method of claim 1, wherein the formation of BRISC dimers can be detected by (i) mass spectrometry and/or (ii) measuring fluorescence signals and/or luminescence signals before and after addition of the compound, wherein for (ii) a relative increase in fluorescence signals and/or luminescence signals indicates an increase in the level of BRISC dimers or an increase in the fluorescence signal of the acceptor together with a decrease in the fluorescence signal of the donor indicates an increase in the level of BRISC dimers.

14. The method of claim 1, wherein an increase in the level of BRISC dimers of about 10% of BRISC after contacting the population of BRISC monomers with the test compound identifies the test compound as a selective BRISC inhibitor.

15. A BRISC dimer comprising:
(i) a first detection label attached to a first BRISC monomer; and
(ii) a second detection label attached to a second BRISC monomer, wherein the first detection label of (i) and the second detection label of (ii) are fused to each other.

16. The BRISC dimer of claim 15, wherein the first detection label and the second detection label are fused to each other by an intermediate molecule.

17. The BRISC dimer of claim 15, wherein (i) the first detection label is beta1-10 of GFP and the second detection label is beta 11 of GFP and the intermediate molecule is a Gly-Ser linker; or (ii) the first detection label is FRB and the second detection label is FKBP and the intermediate molecule is rapamycin.

18. The BRISC dimer of claim 15, wherein each BRISC monomer comprises an octameric complex comprising the subunits, Abraxas2, BRCC36, BRCC45 and MERIT40 at a 2:2:2:2 ratio and the BRISC dimer comprises a 16-meric complex comprising the subunits, Abraxas2, BRCC36, BRCC45 and MERIT40 at a 4:4:4:4 ratio.

19. The BRISC dimer of claim 15, wherein the first detection label is attached to the C-terminus of BRCC45 of the first BRISC monomer and the second detection label is attached the C-terminus of BRCC36 of a second BRISC monomer.

20. A method comprising, using of a BRISC dimer as defined by claim 15 to generate cryo-Electron Microscopy (cryo-EM), crystallography, nuclear magnetic resonance and/or X-ray crystallography structures for structure guided drug design.
